(19) United States
(12) Patent Application Publication
(10) Pub. No.: US 2003/0077247 A1
(43) Pub. Date: Apr. 24, 2003

(54) Chemokines as adjuvants of immune response

(75) Inventors: Christophe Caux, Bressolles (FR); Beatrice Vanbervliet, Dardilly (FR); Carine Paturel, Ecully (FR); Alain Vicari, La Tour de Salvagny (FR); Giorgio Trinchieri, Charly (FR); Francine Briere, Saint Germain sur l'Arbresle (FR); Nathalie Bendriss, Lyon (FR)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/247,395

(22) Filed: Sep. 19, 2002

Related U.S. Application Data
(60) Provisional application No. 60/323,604, filed on Sep. 20, 2001.

(51) Int. Cl. A61K 38/19; A61K 38/20; A61K 39/00; A61K 39/12; A61K 39/02

(52) U.S. Cl. 424/85.1; 424/85.2; 424/185.1; 424/204.1; 424/234.1; 424/274.1

(57) ABSTRACT

Dendritic cells play a critical role in antigen-specific immune responses. Materials and methods are provided for treating disease states, including cancer, infectious diseases, autoimmune diseases, transplantation, and allergy by facilitating or inhibiting the migration or activation of a specific subset of antigen-presenting dendritic cells known as plasmacytoid dendritic cells (pDC). In particular, methods for treating disease states are provided comprising administration of chemokine receptor agonists and antagonists, alone or in combination with a disease-associated antigen, with or without an activating agent.

### FIELD OF THE INVENTION

The invention relates to the use of human chemokine receptor agonists and antagonists in the treatment of disease states, including cancer. The administered chemokine receptor agonists and antagonists direct or prevent the migration of a specific subset of dendritic cells. In one embodiment, disease-specific antigen(s) and/or a moiety designed to activate dendritic cells is administered in conjunction with the chemokine receptor agonist(s).

### BACKGROUND OF THE INVENTION

Dendritic cells (DC) specialize in the uptake of antigen and their presentation to T cells. DC thus play a critical role in antigen-specific immune responses.

DC are bone marrow-derived and migrate as precursors through bloodstream to tissues, where they become resident cells such as Langerhans cells in the epidermis. In the periphery, following pathogen invasion, immature DC such as Langerhans cells are recruited to the site of inflammation (Kaplan et al., 1992, J. Exp. Med. 175:1717-1728; McWilliam et al., 1994, J. Exp. Med. 179:1331-1336) where they capture and process antigens, (Inaba et al., 1986. J. Exp. Med. 164:605-613; Streilein et al., 1989, J. Immunol. 143:3925-3933; Romani et al., 1989, J. Exp. Med. 169:1169-1178; Puré et al., 1990. J. Exp. Med. 172:1459-1469; Schuler et al., 1985, J. Exp. Med. 161:526-546). Antigen-loaded DC then migrate from the peripheral tissue via the lymphatics to the T cell rich area of the lymph nodes, where the mature DC are called interdigitating cells (IDC) (Austyn et al., 1988, J. Exp. Med. 167:646-651; Kupiec-Weglinski et al., 1988, J. Exp. Med. 167:632-645; Larsen et a., 1990, J. Exp. Med. 172:1483-1494; Fossum, S. 1988, Scand. J. Immunol. 27:97-105; Macatonia et al., 1987, J. Exp. Med. 166:1654-1667; Kripke et al., 1990, J. Immunol. 145:2833-2838). At this site, they present the processed antigens to naive T cells and generate an antigen-specific primary T cell response (Liu et al., 1993, J. Exp. Med. 177:1299-1307; Sornasse et al., 1992, J. Exp. Med. 175:15-21; Heufler et al., 1988, J. Exp. Med. 167:700-705).

The DC system is composed of a diverse population of morphologically similar cell types distributed widely throughout the body (Caux et al., 1995, Immunology Today 16:2; Steinman, 1991, Ann. Rev. Immunol. 9:271-296). Some dendritic cells, such as the langerhans cells (LC) of the epidermis, play the role of sentinel of the immune system. Other DC subpopulations, such as monocytes, blood CD11c+ DC, and plasmacytoid DC (pDC), are circulating cells that need to be recruited during infection in specific anatomic sites.

Plasmacytoid DC (pDC) were first characterized by pathologists as plasmacytoid monocytes/T cells accumulating around the HEV of inflamed lymph nodes (Vollenweider et al., 1983, Virchows Arch. (Cell Pathol.) 44:1-114; Facchetti et al., 1988, Hum Pathol 19 (9):1085-92; Facchetti et al., 1988, Am. J. Pathol. 133:15-21). Then, identified as a CD11c− DC subset from blood (O'Doherty et al., 1994, Immunology 82:487-493), they were characterized as plasmacytoid due to their ultrastructural resemblance to Ig-secreting plasma cells upon isolation from tonsils (Grouard et al., 1997, J. Exp. Med. 185(6): 1101-1111). They are characterized by a unique surface phenotype (CD4+IL−3R++CD45RA+HLA−DR+) (Grouard et al., 1997, J. Exp. Med. 185(6): 1101-1111; Facchetti et al., 1999, Histopathology 35(1):88-9; Res et al., 1999, Blood 94 (8):2647-57). It has recently been demonstrated that pDC are identical to natural IFNα producing cells (NIPC) (Siegal et al., 1999, Science 284(5421):1835-7; Cella et al., 1999, Nature Med. 5:919-923), which have long been known as the main source of IFNα in blood in anti-viral immune responses (Ito et al., 1981, Infect Immun 31(2):519-23; Fitzgerald-Bocarsly et al., 1993, Pharmacol. Ther. 60:39-62; Feldman et al., 1994, Virology 204 (1):1-7; (Perussia et al., 1985, Nat Immun Cell Growth Regul 4(3):120-37; Chehimi et al., 1989, Immunology 68(4):488-90; Fitzgerald-Bocarsly et al., 1988, J Leukoc Biol 43(4):323-34; Feldman et al., 1990, J Interferon Res 10(4):435-46). Following virus encounter, these cells produce high levels of IFNα and induce potent in vitro priming and Th-1 polarization of naive T cells (Cella et al., 2000, Nat Immunol 1(4):305-10; Kadowaki et al., 2000, J Exp Med 192 (2):219-26). The origin of pDC is still unclear, but several elements suggest that they may be derived from a precursor common with T cells and B cells: i) they lack expression of myeloid antigens (Grouard et al., 1997, J. Exp. Med. 185, 6:1101-1111; Res et al., 1999. Blood 94, 8:2647-57), ii) they express pre-TCR transcript (Res et al., 1999, Blood 94 (8):2647-57; Bruno et al., 1997, J. Exp. Med. 185:875-884) and SPI-B a lymphoid cells transcription factor (Bendriss-Vermare et al., 2001, JCI 107 :835) iii) development of pDC, T and B, but not myeloid DC is blocked by ectopic expression of inhibitor of DNA binding Id2 or Id3 (Spits et al., 2000, J. Exp. Med. 192 (12):1775-84).

In addition to their morphology, their IFNα production and their putative origin, pDC also differ from myeloid DC in their weak phagocytic activity (Grouard et al., 1997, J. Exp. Med. 185(6):1101-1111), their weak IL-12 production capacity (Rissoan et al., 1999, Science 283:1183-1186), and the signals inducing their activation (Kadowaki et al., 2001, J Immunol 166(4):2291-5). In particular, pDC will respond to CpG but not to LPS activation by producing IFNα, while myeloid DC will mainly respond to LPS by producing IL-12 (Cella et al., 1996, J. Exp. Med. 184:747-752; Koch et al., 1996, J. Exp. Med. 184:741-746). pDC have been shown to induce Th-1 immune responses (Rissoan et al., 1999, Science 283:1183) or Th-2 immune responses (Kadowaki et al., 2000, JEM 192:219), depending on the presence or absence of activation signal (Liu et al., 2001, Nature Immunol 2:585). While recruitment of activated pDC should initiate immunity through naive T cell activation, inactivated DC have been reported to induce immune tolerance, likely through induction of regulatory T cells (Jonuleit et al., 2001, Trends Immunol. 22:394; Bell et al., 2001, Trends Immunol 22:11, Roncarolo et al., 2001, JEM 193:F5; Jonuleit et al., 2000, JEM 162:1213). Moreover, pDC have been shown to induce IL-10 secreting T cells (Rissoan et al., 1999, Science 283:1183; Liu et al., 2001, Nature Immunol 2:585) and CD8 regulatory T cells (Gilliet et al., 2002, J Exp Med. 195(6):695-704). Furthermore, pDC have been recently associated with auto-immune diseases, in particular Lupus (Farkas et al., 2001, Am. J. Pathol. 159:237). In addition, active recruitment of pDC in ovarian tumors has been reported (Curiel et al., 2001, Keystone Symposia March 12-18, 2001: Dendritic Cells, Interfaces With Immunobiology and Medicine), demonstrating that pDC may be favorable to tumor development in certain circumstances, likely through induction of regulatory immune responses. In these cases, the tumor environment is suspected to prevent activation of pDC.

Chemokines are small molecular weight proteins that regulate leukocyte migration and activation (Oppenheim, 1993, Adv. Exp. Med. Biol. 351:183-186; Schall, et al., 1994, Curr. Opin. Immunol. 6:865-873; Rollins, 1997, Blood 90:909-928; Baggiolini, et al., 1994, Adv. Immunol. 55:97-179). They are secreted by activated leukocytes themselves, and by stromal cells including endothelial cells and epithelial cells upon inflammatory stimuli (Oppenheim, 1993, Adv. Exp. Med. Biol. 351:183-186; Schall, et al., 1994, Curr. Opin. Immunol. 6:865-873; Rollins, 1997, Blood 90:909-928; Baggiolini, et a/., 1994, Adv. Immunol. 55:97-179). Responses to chemokines are mediated by seven transmembrane spanning G-protein-coupled receptors (Rollins, 1997, Blood 90:909-928; Premack, et al., 1996, Nat. Med. 2:1174-1178; Murphy, P. M. 1994, Ann. Rev. Immunol. 12:593-633).

It has been shown that several proteins belonging to the chemokine structural family could promote the recruitment of certain subsets of dendritic cells (DC) in vitro (Caux, et al., 2000, Springer Semin Immunopathol. 22:345-69; Sozzani, et al., 1997, J. Immunol. 159:1993-2000; Xu, et al., 1996, J. Leukoc. Biol. 60:365-371; MacPherson, et al., 1995, J. Immunol. 154:1317-1322; Roake, et al., 1995, J. Exp. Med. 181:2237-2247). Signals which regulate the trafficking of dendritic cells, however, are complex and not fully understood. In particular, very little information is available regarding the migratory capacity of plasmacytoid dendritic cells. An understanding of the signals involved in recruitment and migration of this DC subclass would be useful in the development of therapeutics to control or modulate the immune response and to treat immune diseases. In particular, the mobilizations of pDC in tumors would allow exploitation of their function to elicit or amplify anti-tumor immunity. As pDC are key initiators of anti-viral immunity, their controlled manipulation would be expected to result in potent anti-tumor immunity.

There is a continuing need for improved materials and methods that can be used not only to expand and activate antigen presenting dendritic cells, but to modulate the migration of DC so as to be both therapeutically as well as prophylactically useful.

### SUMMARY OF THE INVENTION

The present invention fulfills the foregoing need by providing materials and methods for treating disease states by facilitating or inhibiting the migration or activation of a specific subset of antigen-presenting dendritic cells. It has now been discovered that human plasmacytoid DC (pDC), the natural IFNα producing cells of blood, follow unique trafficking routes controlled by selected chemokines. Thus, administration of specific chemokine receptor agonists or antagonists, alone or in combination with a disease-associated antigen, is a useful therapeutic method. Disease states which can be treated in accordance with the invention include parasitic infections, bacterial infections, viral infections, fungal infections, cancer, autoimmune diseases, graft rejection and allergy.

Thus, the invention provides a method of treating disease states comprising administering to an individual in need thereof an amount of a chemokine receptor agonist or antagonist sufficient to increase or decrease the migration of plasmacytoid dendritic cells to the site of antigen delivery.

The present invention provides a method of treating a disease state comprising administering to an individual in need thereof an amount of a chemokine receptor agonist sufficient to enhance an immune response (through pDC recruitment and activation), wherein the chemokine receptor agonist is selected from the group consisting of a CXCR3 agonist, a CXCR4 agonist, a CCR6 agonist, and a CCR10 agonist, or a combination thereof. Preferably, the disease state is parasitic infection, bacterial infection, viral infection, fungal infection, or cancer. More preferably, the disease state is cancer.

In certain embodiments, the chemokine receptor agonist is a natural ligand selected from the group consisting of SDF-1, IP-10, Mig, I-TAC, CTACK, MEC, Mip-3α, or variants thereof. In certain embodiments, the chemokine receptor agonist is recombinant. In other embodiments, the chemokine receptor agonist is a small molecule. The chemokine receptor agonist(s) can be administered alone or in combination with other chemokine receptor agonist(s).

In a preferred aspect, the chemokine receptor agonist(s) is/are administered with a disease-associated antigen, for instance, in the form of a fusion protein. Such antigens can be tumor associated, bacterial, viral, fungal, or a self antigen, a histocompatability antigen or an allergen.

The chemokine receptor agonist(s) may be administered in the form of a fusion protein comprising one or more chemokine receptor agonists fused to one or more disease associated antigens, or by way of a DNA or viral vector encoding for the chemokine receptor agonist(s) with or without antigens. In preferred embodiments, the chemokine receptor agonist(s) are administered locally and/or systemically.

The chemokine receptor agonist(s) may also be administered in the form of a targeting construct comprising a chemokine receptor agonist and a targeting moiety, wherein the targeting moiety is a peptide, a protein, an antibody or antibody fragment, a small molecule, or a vector such as a viral vector, which is engineered to recognize or target a tumor-associated antigen or a structure specifically expressed by non-cancerous components of the tumor, such as the tumor vasculature. The recognized structure can also be associated with other diseases such as infectious diseases, auto-immunity, allergy or graft rejection.

The chemokine receptor agonist(s) may be administered in combination with a pDC survival factor such as IL-3, IFNα or RANK ligand/agonist.

The chemokine receptor agonist(s) may also be administered in combination with an activating agent such as TNF-α, RANK ligand/agonist, CD40 ligand/agonist or a ligand/agonist of other members of the TNF/CD40 receptor family, IFNα or a TLR ligand/agonist such as CpG.

In one preferred embodiment of the invention, a CXCR3 agonist and a CXCR4 agonist are administered, alone or in combination. Preferably, the CXCR3 agonist is IP-10, Mig, or I-TAC or a variant thereof and the CXCR4 agonist is SDF-1 or a variant thereof. More preferably, the invention provides a method of treating a disease state in an individual in need thereof comprising administering an amount of SDF-1 or a variant thereof in combination with IP-10, Mig, or I-TAC, or a variant thereof. More preferably, a tumor associated antigen or other disease associated antigen is also administered. Most preferably, a survival factor and/or an activating agent is also administered.

In other embodiments of the invention, a CCR6 agonist and/or a CCR10 agonist are administered, alone or in combination. In these embodiments, a survival factor such as IL-3 may be optionally administered. Preferably, the CCR6 agonist is MIP-3α, or a variant thereof and the CCR10 agonist is CTACK or MEC or a variant thereof. Most preferably, a tumor associated antigen, or another disease associated antigen, is also administered. Most preferably, an activating agent is also administered.

In a further embodiment of the invention, a CCR6 agonist and/or a CCR10 agonist is administered in combination with a CXCR3 agonist. In these embodiments, a survival factor such as IL-3 may also be administered. Preferably, the CCR6 agonist is Mip-3α, or a variant thereof, the CCR10 agonist is CTACK, MEC or a variant thereof, and the CXCR3 agonist is selected from the group consisting of IP-10, Mig, I-TAC and variants thereof. The agonists can also be recombinant, or can be in the form of a small molecule. Preferably, a tumor associated antigen or another disease-associated antigen is also administered. Most preferably, an activating agent is also administered.

Another aspect of the invention provides a method for treating disease states comprising administering to an individual in need thereof an amount of a chemokine receptor agonist sufficient to modulate immune response (for instance induce tolerance through induction of regulatory T cells), wherein the chemokine receptor agonist is selected from the group consisting of a CXCR3 agonist, a CXCR4 agonist, a CCR6 agonist, and a CCR10 agonist, or a combination thereof. In these embodiments, chemokine receptor agonist is administered without an activating agent, and the disease state is preferably an autoimmune disease, graft rejection or allergy.

In certain embodiments, the chemokine receptor agonist is a natural ligand selected from the group consisting of SDF-1, IP-10, Mig, I-TAC, CTACK, MEC, Mip-3α, or variants thereof. In certain embodiments, the chemokine receptor agonist is recombinant. In other embodiments, the chemokine receptor agonist is a small molecule. The chemokine receptor agonist(s) can be administered alone or in combination with other chemokine receptor agonist(s).

In a preferred aspect, the chemokine receptor agonist(s) is/are administered with a disease-associated antigen, for instance, in the form of a fusion protein. Such antigens can be a self antigen, a histocompatability antigen or an allergen.

The chemokine receptor agonist(s) may be administered in the form of a fusion protein comprising one or more chemokine receptor agonists fused to one or more disease associated antigens, or by way of a DNA or viral vector encoding for the chemokine receptor agonist(s) with or without antigens. In preferred embodiments, the chemokine receptor agonist(s) are administered locally and/or systemically.

The chemokine receptor agonist(s) may also be administered in the form of a targeting construct comprising a chemokine receptor agonist and a targeting moiety, wherein the targeting moiety is a peptide, a protein, an antibody or antibody fragment, a small molecule, or a vector such as a viral vector, which is engineered to recognize or target a tumor-associated antigen or a structure specifically expressed by non-cancerous components of the tumor, such as the tumor vasculature. The recognized structure can also be associated with other diseases such as infectious diseases, auto-immunity, allergy or graft rejection.

Another aspect of the invention provides a method of treating disease states comprising administering to an individual in need thereof an amount of a chemokine receptor antagonist sufficient to decrease an immune response (by blocking pDC recruitment), wherein the chemokine receptor antagonist is selected from the group consisting of a CXCR3 antagonist, a CXCR4 antagonist, a CCR6 antagonist, and a CCR10 antagonist, or a combination thereof. In these embodiments, the disease state is an autoimmune disease, graft rejection or allergy.

In certain embodiments, the chemokine receptor antagonist is an antagonist of the natural ligand selected from the group consisting of SDF-1, IP-10, Mig, I-TAC, CTACK, and Mip-3α. In certain embodiments, the chemokine receptor antagonist is recombinant. In other embodiments, the chemokine receptor antagonist is a small molecule. The chemokine receptor antagonist(s) can be administered alone or in combination with other chemokine receptor antagonist(s).

The chemokine receptor antagonist(s) may be administered in the form of a fusion protein, or by way of a DNA or viral vector encoding for the chemokine receptor antgonist(s). In preferred embodiments, the chemokine receptor antagonist(s) are administered locally or systemically.

The chemokine receptor antagonist(s) may also be administered in the form of a targeting construct comprising a chemokine receptor antagonist and a targeting moiety, wherein the targeting moiety is a peptide, a protein, an antibody or antibody fragment, a small molecule, or a vector such as a viral vector, which is engineered to recognize or target a structure associated with diseases such as auto-immunity, allergy or graft rejection.

A final aspect of the invention provides a method of treating disease states comprising administering to an individual in need thereof an amount of a chemokine receptor antagonist sufficient to modulate an immune response, wherein the chemokine receptor antagonist is selected from the group consisting of a CXCR3 antagonist, a CXCR4 antagonist, a CCR6 antagonist, and a CCR10 antagonist, or a combination thereof. In these embodiments, the chemokine receptor antagonist is administered without an activating agent, and the disease state is preferably cancer. In particular, the disease state is one in which there is an active recruitment of pDC that may divert the immune response toward regulatory T cells.

In certain embodiments, the chemokine receptor antagonist is an antagonist of the natural ligand selected from the group consisting of SDF-1, IP-10, Mig, I-TAC, CTACK, and Mip-3α. In certain embodiments, the chemokine receptor antagonist is recombinant. In other embodiments, the chemokine receptor antagonist is a small molecule. The chemokine receptor antagonist(s) can be administered alone or in combination with other chemokine receptor antagonist(s).

The chemokine receptor antagonist(s) may be administered in the form of a fusion protein, or by way of a DNA or viral vector encoding for the chemokine receptor antagonist(s). In preferred embodiments, the chemokine receptor antagonist(s) are administered locally or systemically.

The chemokine receptor antagonist(s) may also be administered in the form of a targeting construct comprising a chemokine receptor antagonist and a targeting moiety, wherein the targeting moiety is a peptide, a protein, an antibody or antibody fragment, a small molecule, or a vector such as a viral vector, which is engineered to recognize or target a tumor-associated antigen or a structure spcifically expressed by non-cancerous components of the tumor, such as the tumor vasculature.

### BRIEF DESCRIPTION OF THE DRAWINGS

FIG. 1: pDC express unique pattern of chemokine receptors. pDC were isolated from human blood after magnetic bead depletion of lineage positive cells, and identified based on the triple staining, HLA-DR+, Lineage−, CD11c−.

FIG. 2: pDC do not respond to most inflammatory chemokines.

FIG. 2 shows responses of blood CD11c⁻ pDC and CD11c+ myeloid DC to various chemokines. Each chemokine was tested over a wide range of concentrations (1 to 1000 ng/ml) and only the optimal response is shown. Results are expressed as migration index (ratio chemokine/medium) and represent the mean values obtained from 3 to 10 independent experiments.

FIG. 3: Potent activity of the constitutive chemokine SDF-1 and high CXCR4 expression on pDC. Panel A shows: Dose response to SDF-1 of pDC. Results are expressed as the number of migrating cells and are representative of 5 independent experiments. Panel B shows analysis of: CXCR4 expression on freshly isolated pDC or after 2 hours pre-incubation at 37° C. Results are representative of 5 independent experiments. Panel C shows analysis of: Various DC populations for their response to SDF-1 over a wide range of concentrations (1 to 1000 ng/ml) and only the optimal response is shown. Panel D shows analysis of CXCR4 mRNA by quantitative RT-PCR. Results were normalized using G3PDH as an internal standard, and are expressed as fg/50 ng total RNA. Values represent means from 3 independent samples.

FIG. 4: Human pDC selectively express CXCR3 and at higher levels than other receptors. Panel A shows cell surface expression of CXCR3 on different DC populations, determined by cytofluorimetry. Results are representative of more than 4 independent experiments for each population. Panel B shows CXCR3 mRNA expression on different DC populations determined by quantitative RT-PCR as described in Example 1 and in FIG. 3D. Results were normalized using G3PDH as an internal standard, and are expressed as fg/50 ng total RNA. Values represent means from 3 independent samples. Panel C shows the results of mRNA expression analysis of chemokine receptors on Facs-sorted pDC determined by quantitative RT-PCR as described in Example 1 and in FIG. 3D. Results were normalized using G3PDH as an internal standard, and are expressed as fg/50 ng total RNA. Values represent means from 3 independent samples.

FIG. 5: CXCR3-ligands synergize with SDF-1 to induce potent migration of human pDC. Panel A: Dose response to CXCR3-ligands of pDC in presence or absence of low dose of SDF-1 (20 ng/ml). Panel B: Dose response to SDF-1 of pDC in presence or absence of CXCR3-ligands (1 μg/ml). Results are representative of 3 independent experiments.

FIG. 6: CXCR3-ligands prime human CD11c− plasmacytoid DC by increasing their sensitivity to SDF-1. Panel A shows checkerboard analysis, wherein CXCR4 and CXCR3 ligands were opposed in upper and lower wells. Results are representative of 3 independent experiments. Panel B shows pre-incubation experiments where the cells were first incubated in presence of CXCR4 or CXCR3 ligands for 1 hour before performing the migration assay to both receptor ligands.

FIG. 7: CXCR3 ligands and SDF-1 induce mouse pDC migration FIG. 7 shows response to chemokines in a transwell migration assay of mouse plasmacytoid DC isolated from bone marrow, enriched by magnetic bead depletion and identified based on the triple staining, CD11b−, CD11c+ GR1+. Panel A shows results expressed as migration index (ration chemokine/medium) and represent the mean values obtained from 3 independent experiments. Each chemokine was tested over a wide range of concentrations (1 to 1000 ng/ml) and only the optimal response is shown. Panel B shows the dose response curves of a representative experiment.

FIG. 8: Compared to other DC populations, pDC express high levels of L-selectin, but they also express CLA. Results are representative of more than 4 independent experiments for each population.

FIG. 9: CCR6 and CCR10 expressions are induced on human plasmacytoid DC upon culture in IL-3.

Plasmacytoid DC isolated by Facs-sorting, were cultured in presence of IL-3 for 24 to 96 hours. CCR6 and CCR10 expression was followed by cytofluorimetry at the indicated time points.

FIG. 10: Plamacytoid DC migrate in response to CCL20/MIP-3α only following culture in IL-3 while they acquire CCR10-ligands responsiveness in response to different survival factors.

Plasmacytoid DC isolated by Facs-sorting, were cultured for 48 hours in presence of IL-3, PFA inactivated influenza virus, ODN. Panel A shows CCR6 chemokine receptor expression and migration in transwell migration assays in response to CCL20/MIP-3α. Panel B shows CCR10 chemokine receptor expression and migration in transwell migration assays in response to CCL27/CTACK and CCL28/MEC.

FIG. 11: Upon contact with virus, pDC acquire CCR7 expression and CCR7 ligand activity.

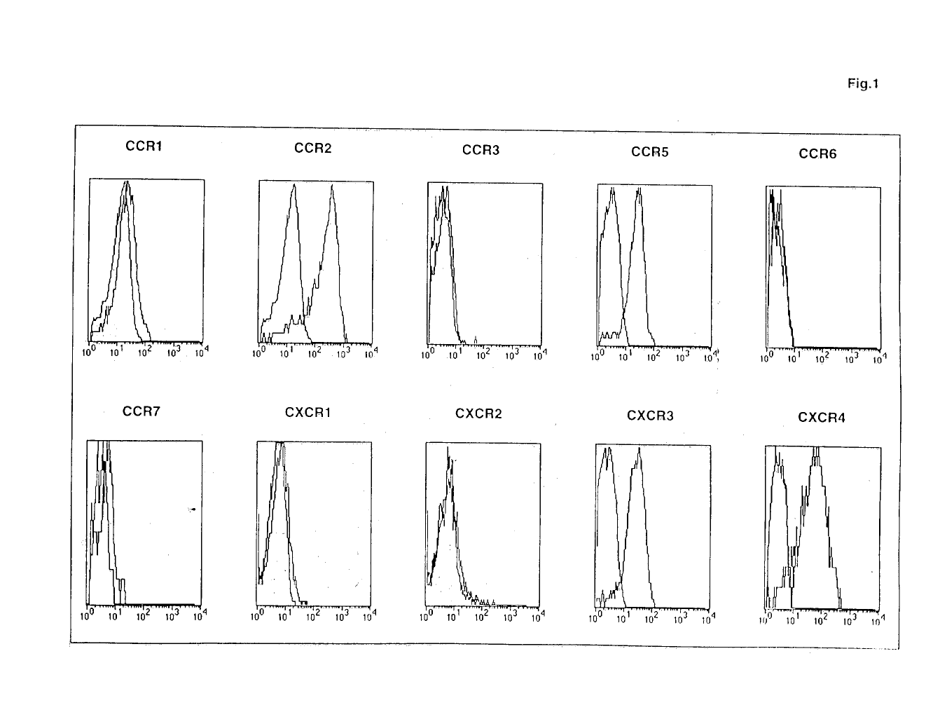

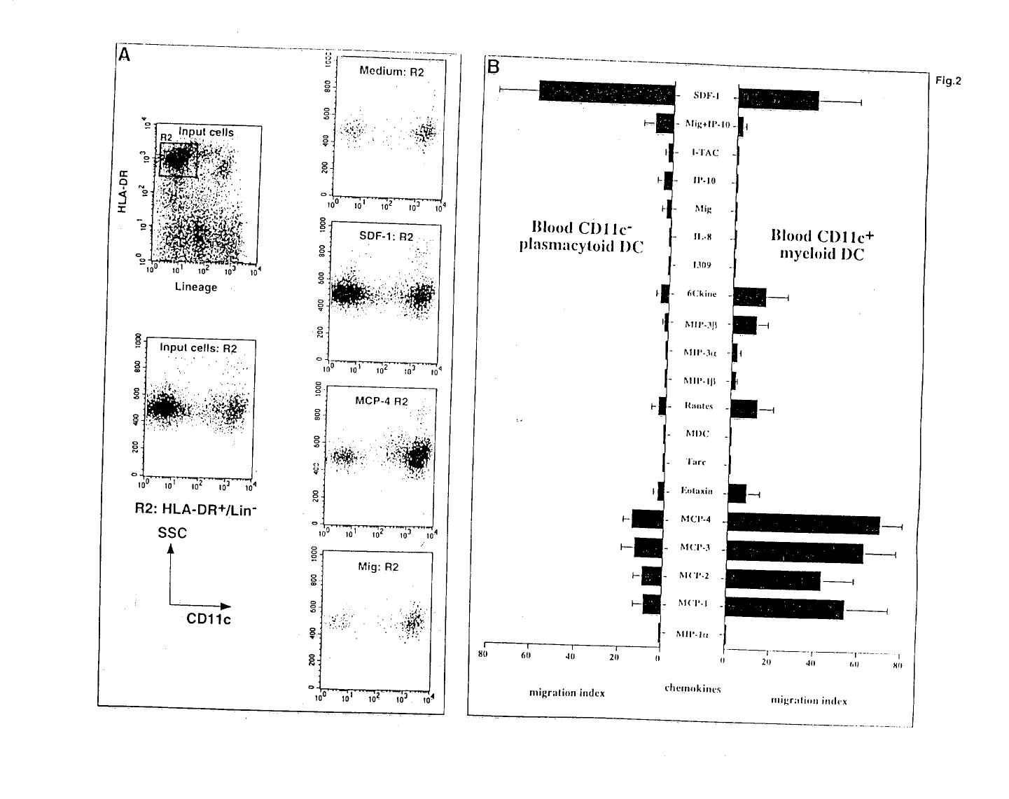

pDC were cultured in medium alone or in presence of PFA inactivated influenza virus (1 hameglutin unit/ml) for 2 hours. Then cells were processed as in FIG. 1 for chemokine receptor expression (panel A) and as in FIG. 2 for chemokine responsiveness (panel B). Results are representative of 3 independent experiments.

### DETAILED DESCRIPTION OF THE INVENTION

All references cited herein are incorporated in their entirety by reference.

The present invention is based in part on the discovery that plasmacytoid dendritic cells (pDC) follow unique trafficking routes as compared to other DC subsets, and that these trafficking routes are regulated by a combination of specific chemokines. The inventors have shown that pDC display a different spectrum of chemokine receptor expression as compared to other DC subsets or precursor populations, and respond to unique chemokine combinations. Based on this discovery, the inventors provide methods of modulating the recruitment of pDC by administration of agonists or antagonists of these receptors, alone or in combination with a disease associated antigen, a pDC survival factor, and/or an activating agent. In view of the key role of pDC in initiating anti-viral immunity, these methods will be useful to achieve potent therapeutic immunity in diseases such as cancer.

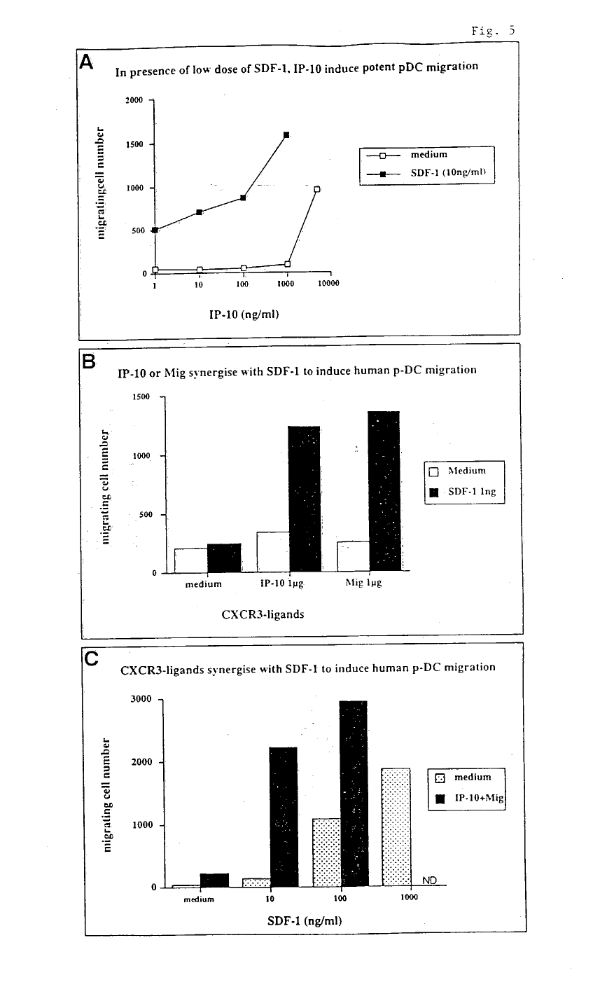

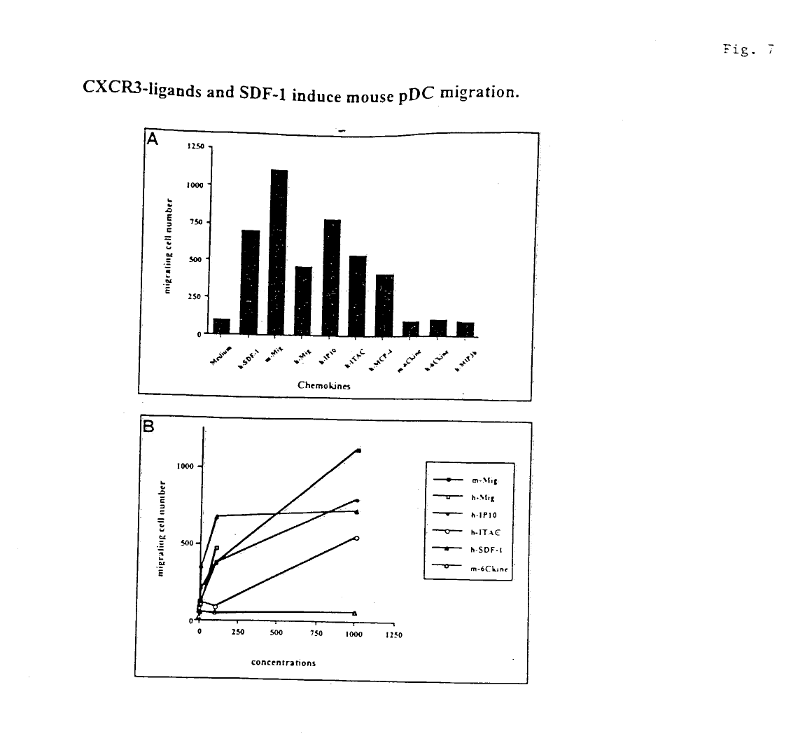

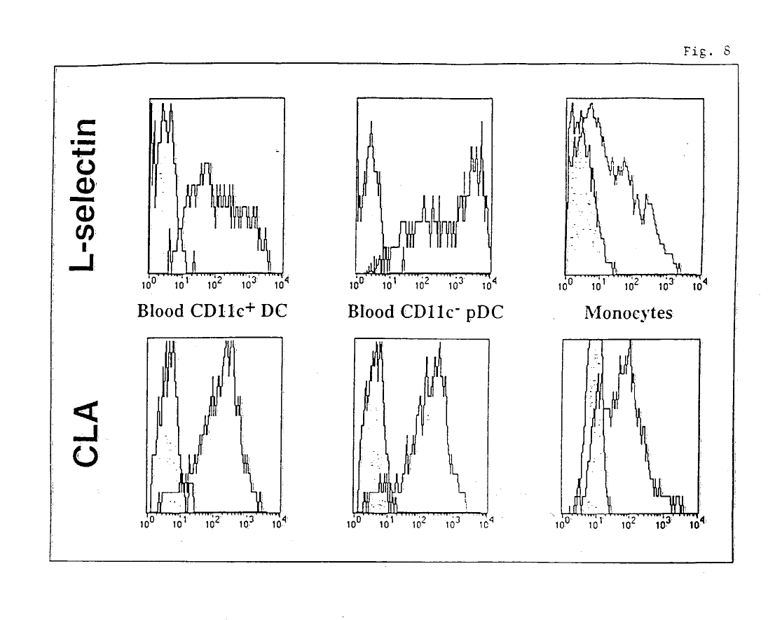

The inventors demonstrate herein that while pDC do not respond to most inflammatory chemokines, the CXCR4 ligand SDF-1 and the CXCR3 ligands Mig, IP-10 and I-TAC are very potent in inducing pDC migration (Examples 1, 3 and 5). Importantly, the inventors have demonstrated that CXCR3 ligands synergize with SDF-1 to induce human pDC migration by decreasing the threshold of sensitivity to SDF-1 (Examples 3 and 4). Furthermore, it is shown that the activity of CXCR3 ligands is independent of a gradient and act by priming the pDC to respond to low SDF-1 concentrations (Example 4, FIG. 8). It is also demonstrated that both human (Examples 1 and 3; FIGS. 2 and 5) and mouse pDC (Example 5; FIG. 7) respond to CXCR3 and CXCR4 ligands. pDC also express the cutaneous homing molecule CLA, suggesting a capacity to enter peripheral skin inflammatory sites (Example 6). Furthermore, in vivo analysis of chemokine expression reveals that, at sites of inflammation, CXCR3 ligands are expressed by endothelial cells in contact with basal epithelial cells expressing SDF-1 (Example 8), arguing for a sequential effect: CXCR3 ligands first, and SDF-1 second for pDC recruitment.

Thus, the inventors have provided methods to selectively recruit pDC comprising administering to an individual in need there of an effective amount of a CXCR3 agonist (which are highly selective for pDC) in combination with a CXCR4 agonist (which are less selective, but are potent chemoattractants). Furthermore, as the activity of CXCR3 ligands can be at least in part gradient independent, (see Example 4 and FIG. 8), these observations suggest that systemic use of CXCR3 agonists in combination with local delivery of CXCR4 agonists would be highly effective in enhancing an immune response. If blocking pDC recuitment is desired, CXCR3 antagonists and CXCR4 antagonists may be administered according to the invention.

It had been previously observed that the migration of myeloid DC required sequential and complementary chemokine gradients; in particular, CCR2+/CCR6− circulating blood DC or precursors are recruited by CCR2-ligands from blood to tissues (Vanbervliet et al., 2001, Eur J Immunol. 32(1):231-42.). Thus, depending on the microenvironment, other receptors might be upregulated (e.g. CCR6 by TGF-β) allowing cells to reach the site of pathogen entry (e.g. skin or mucosa).

In order to better understand the different steps of pDC migration, the inventors have investigated the effects of known key regulators of pDC physiology, in particular the survival factor IL-3, on chemokine receptor expression. It has been concluded that pDC under these conditions express high levels of CCR6 and CCR10, and respond to the chemokine MIP-3α (Example 7). As IL-3 is a survival factor for pDC, it is likely that in vivo, CCR6 and CCR10 expression on pDC represent a physiological step of pDC differentiation. In these conditions, CXCR3 is still highly expressed, suggesting that CXCR3 agonists would be able to synergize with CCR6/CCR10 agonists. Furthermore, in vivo analysis of chemokine expression reveals that, at site of inflammation, CXCR3-ligands, SDF-1, CTACK and MIP-3 cc form complementary gradients, suggesting the sequential action of chemokines for pDC to reach the site of pathogen entry. CXCR3-ligands are expressed by endothelial cells in contact with basal epithelial cells expressing SDF-1 and CTACK (Morales et al., 1999, PNAS 96:14470) and MIP-3α is expressed by the outer-layer of the epithelium (Example 8).

Therefore, in addition to the methods decribed above, the invention also provides methods for treating disease states in which enhancing or modulating an immune response is desirable comprising administering to an individual in need thereof an amount of a CCR6 agonist and/or a CCR10 agonist, alone or in combination with a survival factor such as IL-3 or other factors inducing these receptors. The CCR6 agonists and CCR10 agonists may also be administered in combination with CXCR3 agonists and CXCR4 agonists. The specific activity of CCR6 and CCR10 ligands on this unique cell type also allow the use of CCR6/CCR10 antagonists (with or without CXCR3/CXCR4 antagonists) in pathologies such as auto-immunity, allergy and transplantation, but also in some types of tumors and infectious diseases.

Finally, upon contact with viruses, pDC very rapidly up-regulate expression of CCR7 and acquire CCR7 ligand responsiveness (see Example 9), suggesting that following local recruitment and activation, these cells will have the capacity to emigrate in the lymph node through the lymphatic stream, a process controlled by CCR7 and its ligands (Sallusto et al., 2000, Immunol. Rev 177:134; Sozzani et al., 2000, JCI 20:151). Thus, combination of chemokine receptor agonists allowing pDC recruitment, together with signals inducing pDC activation, will empower pDC to emigrate to the lymph node through the lymphatic stream, and to induce immune responses in the lymph nodes.

Depending on their state of activation, pDC have been shown to induce Th-2 immune responses (Rissoan et al., 1999, Science 283:1183) or Th-1 immune responses (Kadowaki et al., 2000, JEM 192:219; Liu et al., 2001, Nature Immunol 2:585). Thus, depending on the context, agonists and antagonists of chemokine receptors which are selectively expressed on pDC might be used to either induce or suppress pDC migration in order to modulate immunity.

Thus, one application of the discoveries set forth herein are methods for using agonists of these pDC specific receptors to enhance the immune response by recruiting pDC and activating them, as desired in the case of cancer and infectious diseases. In this context, the goal is to recruit and activate pDC to the site of antigen expression, and these methods may optionally include administration of a survival factor and/or an activating agent which promotes pDC survival or empowers them to initiate immunity through naive T cell activation.

In other circumstances, chemokine receptor agonists can also be used to induce immune tolerance. Inactivated DC have been reported to induce immune tolerance, likely through induction of regulatory T cells (Jonuleit H., 2001, Trends Immunol 22:394; Bell E., 2001, Trends Immunol 22:11; Roncarolo M. G., 2001, JEM 193:F5; Jonuleit H., 2000, JEM 162:1213). Moreover, pDC have been shown to induce IL-10 secreting T cells (Rissoan M. C., 1999, Science 283:1183; Liu Y. J., 2001, Nature Immunol 2:585) and CD8 regulatory T cells (Gilliet et al. IL-10-producing CD8+T suppressors Cells induced by Plasmacytoid-derived DC, Submitted). Thus, the present invention also provides methods for using chemokine receptor agonists to decrease the immune response, as would be desirable in the case of autoimmunity, allergy and transplantation. In this context, the goal is to recruit inactivated pDC; therefore, these methods do not include administration of an activating agent.

Likewise, chemokine receptor antagonists can be used to treat different disease states. In disease states such as autoimmunity, allergy and transplantation, antagonists can be used to decrease the recruitment of activated pDC. As an example, pDC have been recently associated with auto-immune diseases, in particular Lupus (Farkas et al., 2001, Am. J. Pathol. 159:237). However, antagonists can also be used in certain cancers where blocking pDC recruitment would be desirable. For example, active recruitment of pDC in ovarian tumors has been reported (Curiel et al., Kestone Symposia March 12-18 2001: Dendritic cells, interfaces with immunobiology and medicine), demonstrating that pDC may be favorable to tumor development in certain circumstances, likely through induction of regulatory immune responses. In these cases, the tumor environment is suspected to prevent activation of pDC. Thus, methods for treating these disease states comprising administering chemokine receptor antagonists would be applicable.

Thus, the chemokine receptor agonists and antagonists described herein can be used in accordance with the invention to selectively induce or suppress pDC recruitment. Combinations of CXCR3, CXCR4, CCR6 and/or CCR10 agonists and survival factors, with or without a disease associated antigen, with or without an activating agent, can be used to treat disease states in which enhancing or modulating an immune response is desirable. Combinations of CXCR3, CXCR4, CCR6 and/or CCR10 antagonists can be used when blocking pDC function by interfering with pDC migration is desirable.

The chemokine receptor CXCR4 (NPY3R) is a coreceptor with CD4 (186940) for T-lymphocyte cell line tropic human immunodeficiency virus type 1 (HIV-1) (Feng et al., 1996, Science 272:1955-58). It has been found to be highly expressed in primary and metastatic human breast cancer cells but is undetectable in normal mammary tissue(Muller et al., 2001, Nature 410:6824). Histologic and quantitative PCR analyses showed that metastasis of intravenously or orthotopically injected breast cancer cells could be significantly decreased in SCID mice by treatment with anti-CXCR4 antibodies.

Stromal cell-derived factors 1-alpha and 1-beta (SDF1) (Swiss-prot accession number P30991) is the principal ligand for CXCR4 (Nishikawa et al., 1988, Eur. J. Immunol. 18(11):1767-71). The mouse SDF-1 alpha and beta proteins are identical in the 89 N-terminal amino acids but the beta form has an additional 4 residues at the C-terminus. Swiss prot accession number P30991. Human SDF-1 bears approximately 92% identity to the mouse proteins (Shirozu et al., 1995, Genomics 28(3):495-500). The human alpha and beta isoforms are a consequence of alternative splicing of a single gene; the alpha form is derived from exons 1-3 while the beta form contains additional sequence from exon 4. SDF1 has been shown to be a highly efficacious lymphocyte chemoattractant (Bleul et al., 1996, J. Exp. Med 184(3):1101-9; Bleul et al., 1996, Nature 382(65994):829-33).

CXCR3 is a chemokine receptor whose expression is limited to IL-2 and active T lymphocytes (see WO 98/11218, published Mar. 19, 1998). Known CXCR3 ligands include IP-10, Mig and I-TAC. CXCR3 has been shown to be preferentially expressed by Th-1 cells (Campbell et al., 2000, Arch. Immunol. Ther. Exp. 48:451-6) and NK cells (Taub et al., 1995, J. Immunol. 164:3112-22). CXCR3 ligands have anti-angiogenic activity, and represent the ultimate mediator in the anti-tumor action of a cytokine cascade involving IL-12 and IFNα (Narvaiza et al., 2000, J. Immunol. 164:3112-22; Sgadari et al., 1996, Blood 87:3877-82; Kanegane et al., 1998, J. Leukoc. Biol. 64:384-92).

IP-10 (CXCL10, Swiss-Prot accession number P02778 for human protein), Mig (CXCL9, Swiss-Prot accession number Q07325 for human protein), and I-TAC (CXCL11, Swiss-Prot accession number 014625 for human protein) are 3 ligands for CXCR3 (Farber et al., 1997, J. Leukoc. Biol. 61:246-57; Cole et al., 1998, J. Exp. Med. 187:2009-21). IP-10 and Mig were initially reported as IFNγ induced genes (Cole et al., 1998, J. Exp. Med. 187:2009-21; Luster et al., 1987, J. Exp. Med. 166:1084-97; Farber et al., 1990, Nat'l Acad. Sci. 87:5238-42). IP-10 and Mig are induced upon viral challenge (Salazar-Mather et al., 2000, J. Clin. Invest. 105:985-93) and can also be expressed in absence of IFNγ (Mahalingam et al., 2001, JBC 276:7568).

The chemokine receptor CCR6 is expressed by 40-50% of peripheral blood memory, but not naive, T cells, in particular in T cells with epithelial homing properties (See WO98/01557; Fitzhugh et al., 2000, J. Immunol. 165:6677-6681). The ligand for CCR6, MIP-3α, has also been known as LARC, exodus and CCL20 (Fitzhugh et al., 2000, J. Immunol. 165:6677-6681). MIP-3α is one of a small number of chemokines including SDF-1, 6Ckine and TARC that have been demonstrated to induce arrest of lymphocytes under physiologic flow conditions (Campbell et al., 1998, Science 279:381; Campbell et al., 1999, Nature 400:776; Tangemann et al., 1998, J. Immunol. 161:6330). The amino acid sequence of MIP-3 alpha can be found in accession U77035.1, Rossi et al., 1997, J. Immunol. 158:1033. Among DC populations, CCR6/MIP-3α has been reported to be selectively involved in skin Langerhans cells migration (Dieu et al., 1998, J. Exp. Med 188(2):373-86; Dieu-Nosjean et al., 2000, J. Exp. Med. 192(5):705-18; Charbonnier et al, 1999, J. Exp. Med., 190(12):1755-68), as well as on subsets of epithelial DC of the gut (Iwasaki et al, 2000, J. Exp. Med. 191(8):1381; Cook et al., 2000, Immunity 12(5)495-503. Furthermore, in vivo Mip-3α expression is restricted to inflamed epithelium (Dieu et al., 1998, J. Exp. Med 188(2):373-86; Dieu-Nosjean et al., 2000, J. Exp. Med. 192(5):705-18; Tanaka et al., 1999, Eur. J. Immunol. 29(2):633-42).

The chemokine receptor CCR10 is disclosed in Bonini et al., 1997, DNA Cell Biol. 16(10):12499-56. Known CCR10 ligands include the chemokine CTACK/CCL27 (Swiss-prot accession number Q9Y4X3), a skin-associated chemokine that preferentially attracts skin-homing memory T cells (Morales et al., 1999,. Proc. Natl. Acad. Sci. USA 96:14470; Homey et al., 2000, J. Immunol. 164(7):3465-70). More recently, the mucosae-associated epithelial chemokine (MEC/CCL28) (swissprot accession number Q9NRJ3), which is expressed in diverse mucosal tissues, has been identified as a novel chemokine ligand for CCR10 (Pan et al., 2000, The Journal of Immunology, 2000, 165:2943-2949).

A “chemokine receptor agonist” for use in the invention is an agent that is active on a restricted subset of DC, in particular pDC, through a receptor expressed on pDC, such as the CXCR3, CXCR4, CCR6 or CCR10 receptor. The term encompasses natural proteins of the body such as chemokine ligands of the CXCR3, CXCR4, CCR6 and CCR10 receptors. Several of these chemokines, including, but not limited to, IP-10, Mig, I-TAC, SDF-1, MIP-3α, CTACK/CCL27 and MEC/CCL28 have been identified by the inventors. In addition to the chemokines disclosed herein, other CXCR3, CXCR4, CCR6 and CCR10 ligands can be used in the methods of the invention. The term also includes variants of said chemokines. Such variants will continue to possess the desired pDC chemoattractant activity discussed above. Variants refers to a polypeptide derived from the native protein by deletion or addition of one or more amino acids to the N-terminal and/or C-terminal end of the native protein; deletion or addition of one or more amino acids at one or more sites in the native protein; or substitution of one or more amino acids at one or more sites in the native protein. Such variants include mutants, fragments, allelic variants, homologous orthologs, and fusions of native protein. Chemokine receptor agonists may also be modified by glycosylation, phosphorylation, substitution of non-natural amino acid analogs and the like.

In addition, ligand screening using CXCR3, CXCR4, CCR6 and CCR10 receptors or fragments thereof can be performed to identify molecules having binding affinity to the receptors. Subsequent biological assays can then be utilized to determine if a putative agonist can provide activity. If a compound has intrinsic stimulating activity, it can activate the receptor and is thus an agonist in that it stimulates the activity of the receptor or mimics the activity of the ligand, e.g., inducing signaling.

Chemokine receptor agonists which are small molecules may also be identified by known screening procedures. In particular, it is well known in the art how to screen for small molecules which specifically bind a given target, for example tumor-associated molecules such as receptors. See, e.g., Meetings on High Throughput Screening, International Business Communications, Southborough, Mass. 01772-1749.

A “chemokine receptor antagonist” for use in the invention is an agent that decreases the migration of a restricted subset of DC, in particular pDC, by blocking the activity of the CXCR3, CXCR4, CCR6 or CCR10 receptor. The term includes both antagonists of the receptor(s) and antagonists of the ligand(s). A chemokine receptor antagonist of the invention can be derived from antibodies or comprise antibody fragments. In addition, any small molecules antagonists, antisense nucleotide sequence, nucleotide sequences included in gene delivery vectors such as adenoviral or retroviral vectors that decrease the migration of pDC would fall within this definition. Similarly, soluble forms of the CXCR3, CXCR4, CCR6 and CCR10 receptor lacking the transmembrane domains can be used. Finally, mutant antagonist forms of the natural ligands can be used which bind strongly to the corresponding receptors but essentially lack biological activity.

Various other chemokine receptor antagonists can be produced. Receptor binding assays can be developed. See, e.g. Bieri et al., 1999, Nature Biotechnology 17:1105-1108, and accompanying note on page 1060. Calcium flux assays may be developed to screen for compounds possessing antagonist activity. Migration assays may take advantage of the movement of cells through pores in membranes, which can form the basis of antagonist assays. Chemotaxis may be measured thereby. Alternatively, chemokinetic assays may be developed, which measure the induction of kinetic movement, not necessarily relative to a gradient, per se.

Chemokine receptor antagonists which are small molecules may also be identified by known screening procedures. In particular, it is well known in the art how to screen for small molecules which specifically bind a given target, for example tumor-associated molecules such as receptors. See, e.g., Meetings on High Throughput Screening, International Business Communications, Southborough, Mass. 01772-1749.

A “survival factor” for use in the invention is defined as an agent which provides signals which promote survival of pDC and are permissive for a pDC differentiation program, including appearance of skin homing properties and chemokine receptor expression. Examples of survival factors include but are not limited to natural products of the body such as IL-3, or IFNα and RANK ligand, which are survival factors for pDC without inducing their maturation.

An “activating agent” for use in the invention is defined as a moiety that is able to activate, induce or stimulate maturity of pDC. Such agents provide maturation signals which promote migration from the tissues to the lymph nodes and empower pDC to activate naive T cells. Examples of activating agents include but are not limited to a natural product of the body such as IFNα, TNF-α, RANK ligand, CD40 ligand or a ligand of other members of the TNF/CD40 receptor family, or an agonist antibody recognizing a specific structure on DC such as an anti-CD-40/RANK antibody, or another substance. The activating substance can also be a sequence of nucleic acids containing unmethylated CpG motifs or agonist of a toll-like receptor known to stimulate DC. In the embodiment of the invention where the chemokine receptor agonist/antagonist and/or antigen is delivered by the means of a plasmid vector, these nucleic acid sequences may be part of the vector.

A chemokine receptor agonist or antagonist described above may be administered alone or in combination with one or more additional chemokine receptor agonist or antagonist. The chemokine receptor agonist/antagonist can by delivered or administered at the same site or a different site (systemic versus local), and can be administered at the same time as one or more other chemokine receptor agonist or antagonist, or after a delay not exceeding 48 hours. Concurrent or combined administration as used herein means the chemokine and antigen are administered to the subject either (a) simultaneously in time, or (b) at different times during the course of a common treatment schedule. In the latter case, the two compounds are administered sufficiently close in time to achieve the intended effect.

The mode of delivery of the various chemokine receptor agonists and chemokine receptor antagonists may be by injection, including intradermal, intramuscular, intratumoral, subcutaneous, intra-venous or per os, or topical, such as an ointment or a patch.

The chemokine receptor agonists/antagonists may also be delivered as a nucleic acid sequence by the way of a vector, such as a viral vector (e.g., adenovirus, poxvirus, retrovirus, lentivirus), or an engineered plasmid DNA.

The chemokine receptor agonists/antagonists may be administered alone or combined with substances allowing for their slow release at delivering site (depot). The chemokine receptor agonists/antagonists may be administered locally or systemically.

The chemokine receptor agonists/antagonists may also be administered as part of a targeting construct comprising a chemokine receptor agonist or antagonist and a targeting moiety designed to recognize or target a disease-associated antigen such as a tumor associated antigen or a structure specifically expressed by non-cancerous components of a tumor, such as the tumor vasculature. Examples of targeting moieties include but are not limited to peptides, proteins, small molecules, vectors, antibodies or antibody fragments (See, e.g., Melani et al., 1998, Cancer Res. 58:4146-4154).

In a particularly preferred embodiment of the invention, the chemokine receptor agonist or chemokine receptor antagonists is administered with a disease-associated antigen. The antigen can be any molecular moiety against which an increase or decrease in immune response is sought. This includes antigens derived from organisms known to cause diseases in man or animal such as bacteria, viruses, parasites and fungi. This also includes antigens expressed by tumors (tumor-associated antigens) and plant/food antigens (allergens), as well as self antigens (autoimmunity).

Tumor associated antigens for use in the invention include, but are not limited to Melan-A, tyrosinase, p97, β-HCG, GaINAc, MAGE-1, MAGE-2, MAGE-3, MAGE-4, MAGE-12, MART-1, MUC1, MUC2, MUC3, MUC4, MUC18, CEA, DDC, melanoma antigen gp75, HKer 8, high molecular weight melanoma antigen, K19, Tyr1 and Tyr2, members of the pMel 17 gene family, c-Met, PSA, PSM, α-fetoprotein, thyroperoxidase, gp100, NY-ESO-1, telomerase and p53. This list is not intended to be exhaustive, but merely exemplary of the types of antigen which may be used in the practice of the invention.

Different combinations of antigens may be used that show optimal function with different ethnic groups, sex, geographic distributions, and stage of disease. In one embodiment of the invention at least two or more different antigens are administered in conjunction with the administration of chemokine.

In addition, a fusion protein consisting of a chemokine receptor agonists such as IP-10, Mig, I-TAC, MIP-3α, CTACK, SDF-1 or a portion thereof and an antigen may be administered.

Both primary and metastatic cancer can be treated in accordance with the invention. Types of cancers which can be treated include but are not limited to those affecting: Oral cavity and pharynx (tongue, mouth, pharynx, others), disgestive system (eosphagus, stomach, small intestine, colon, rectum, anus/anorectum, liver/intrahepatic bile duct, gallbladder/other biliary, pancreas, others), respiratory system (larynx, lung/bronchus, others), head and neck, bones and joints, soft tissues (including heart), skin (basal and squamous carcinoma, melanoma, others), breast, genital system (uterine cervix, uterine corpus, ovary, vulva, vagina, prostate testis, penis, others), urinary system (urinary bladder, kidney/renal pelvis, ureter, others), eye and orbit, brain and nervous system, endocrine system (thyroid, others), blood/hematopoietic system (Hodgkin's lymphoma, non-Hodgkin's lymphoma, multiple myeloma, acute lymphocytic leukemia, chronic lymphocytic leukemia, acute myeloid leukemia, chronic myeloid leukemia, other leukemia). Cancers can be of different cellular origin (for example carcinoma, melanoma, sarcoma, leukemia/lymphoma, etc.) and can be of any known or unknown ethiology (for example sun's rays, viruses, tobacco/alcohol use, profession, nutrition, lifestyle, etc.) The term “carcinoma” refers to malignancies of epithelial or endocrine tissues including respiratory system carcinomas, gastrointestinal system carcinomas, genitourinary system carcinomas, prostatic carcinomas, endocrine system carcinomas. Metastatic, as this term is used herein, is defined as the spread of tumor to a site distant from the primary tumor including regional lymph nodes.

A survival factor or other moiety designed to induce chemokine receptor expression on pDC may be advantageously administered.

An activating agent or other moiety designed to activate, induce or stimulate maturity of pDC may also be administered.

Generally, chemokine(s) and/or antigen(s) and/or survival factor(s)/activating agent(s) and/or cytokine(s) are administered as pharmaceutical compositions comprising an effective amount of chemokine(s) and/or antigen(s) and/or activating agent(s) and/or cytokine(s) in a pharmaceutical carrier. These reagents can be combined for therapeutic use with additional active or inert ingredients, e.g., in conventional pharmaceutically acceptable carriers or diluents, e.g., immunogenic adjuvants, along with physiologically innocuous stabilizers and excipients. A pharmaceutical carrier can be any compatible, non-toxic substance suitable for delivering the compositions of the invention to a patient.

The quantities of reagents necessary for effective therapy will depend upon many different factors, including means of administration, target site, physiological state of the patient, and other medicants administered. Thus, treatment dosages should be titrated to optimize safety and efficacy. Animal testing of effective doses for treatment of particular cancers will provide further predictive indication of human dosage. Various considerations are described, e.g., in Gilman et al. (eds.) (1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa. Methods for administration are discussed therein and below, e.g., for intravenous, intraperitoneal, or intramuscular administration, transdermal diffusion, and others. Pharmaceutically acceptable carriers will include water, saline, buffers, and other compounds described, e.g., in the Merck Index, Merck & Co., Rahway, N.J. Slow release formulations, or a slow release apparatus may be used for continuous administration.

Dosage ranges for chemokine receptor agonist(s) and antagonist(s) and/or antigen(s) and/or survival factor(s) and/or activating agent(s) would ordinarily be expected to be in amounts lower than 1 mM concentrations, typically less than about 10 μM concentrations, usually less than about 100 nM, preferably less than about 10 pM (picomolar), and most preferably less than about 1 fM (femtomolar), with an appropriate carrier. Generally, treatment is initiated with smaller dosages which are less than the optimum dose of the compound. Thereafter, the dosage is increased by small increments until the optimum effect under the circumstance is reached. Determination of the proper dosage and administration regime for a particular situation is within the skill of the art.

Preferred embodiments consist of but are not restricted to administration of a recombinant IP-10, Mig, or I-TAC protein alone, or together with SDF-1, optionally in combination with a survival factor and/or activating agent or combined with substances allowing for its slow release at delivering site (depot); fusion proteins consisting of IP-10, Mig or I-TAC, or a fraction of IP-10, Mig or I-TAC and an antigen (peptide more than 9 amino acids or protein or other antigenic moiety); DNA or viral vector encoding for IP-10, Mig or I-TAC or fraction of IP-10, Mig or I-TAC with or without an antigen (peptide more than 9 amino acids or protein or other antigenic moiety), or a nucleic acid sequence included in a delivery vector. Other preferred embodiments include administration of a recombinant MIP-3α, CTACK or MEP protein, in combination with a survival factor or activating agent, alone or combined with substance allowing for its slow release. In all preferred embodiments, the chemokine receptor agonists can be administered in combination with antigen, with or without an activating agent.

### EXAMPLES

The invention can be illustrated by way of the following non-limiting examples, which can be more easily understood by reference to the following materials and methods.

Hematopoietic Factors, Reagents and Antibodies.

rhGM-CSF (specific activity: 2.106 U/mg, Schering-Plough Research Institute, Kenilworth, N.J.), rhTNFα (specific activity: 2×10⁷ U/mg, Genzyme, Boston, Mass.) rhSCF (specific activity: 4×10⁵ U/mg, R&D Systems, Abington, UK), and rhIL-4 (specific activity: 2.107 U/mg, Schering-Plough Research Institute, Kenilworth, N.J.) were used at the optimal concentrations of 100 ng/ml, 2.5 ng/ml, 25 ng/ml, and 50 U/ml, respectively. Recombinant human chemokines were from R&D Systems and were used at optimal concentration: MCP1/CCL2 (10 ng/ml), MCP2/CCL8 (100 ng/ml), MCP3/CCL7 (100 ng/ml), MCP4/CCL13 (1 μg/ml), MIP3α/CCL20 (1 μg/ml), RANTES/CCL5 (10 ng/ml), MIP1α/CCL3 (10 ng/ml), MIPβ/CCL4 (100 ng/ml), MIP16/CCL15 (100 ng/ml), Eotaxin/CCL11(1 μg/ml), TARC/CCL17 (10 ng/ml-1 μg/ml), MDC/CCL22 (10 ng/ml-1 μg/ml), MIP3β/CCL19 (1 μg/ml), 6Ckine/CCL21 (1 μg/ml), I309/CCL1 (10 ng/ml-1 μg/ml), IL8/CXCL8 (10 ng/ml-1 μg/ml), IP10/CXCL10 (10 ng/ml-1 μg/ml), MIG/CXCL9 (10 ng/ml-1 μg/ml), SDF1α CXCL12 (100 ng/ml) and fractalkine/CX3CL1 (10 ng/ml). Specific PE-conjugated anti-human CCR3 (clone 61628.111) was purchased from R&D Systems. PE anti-human CXCR4 (clone 51505.111), CCR5 (clone 2D7), CCR6 (clone 11A9), and CXCR3 (clone 1C6) were obtained from Pharmingen (San Diego, Calif.). Biotin coupled anti-human CCR1 (clone 53504.111) and CCR2 (clone 48607.211) from R&D Systems, were revealed by PE-conjugated streptavidin (DAKO). Anti-CCR7 (clone 2H4) was a mouse IgM monoclonal antibody (Pharmingen) revealed by biotin coupled goat anti-mouse IgM (Caltag). All antibodies were first validated for their specificity on different blood cell subsets. PE-conjugated anti-CD83 was from Immunotech, and anti-IL-3Ra, anti-CLA, and anti-CD62L were from Pharmingen.

Enrichment for CD11c− Plasmacytoid DC and CD11c⁺ Myeloid DC from Peripheral Blood.

Circulating blood CD11c− plasmacytoid DC (pDC) and myeloid CD11c+ DC were prepared from peripheral blood as previously described (Grouard et al., 1997, J. Exp. Med. 185 (6):1101-1111; Grouard et al., 1996, Nature 384:364-367). Briefly, peripheral blood mononuclear cells were isolated by Ficoll-Hypaque and lineage positive cells were removed using antibodies anti-CD3 (OKT3), anti-CD19 (4G7), anti-CD14 (MOP9), anti-CD56 (NKH1, Coulter), anti-CD16 (ION16, Immunotech), anti-CD35 (CR1, Immunotech), and anti-glycophorin A (JC159, DAKO) and magnetic beads (anti-mouse Ig-coated Dynabeads, Dynal). All the procedures of depletion and staining were performed in presence of 0.5 mM EDTA. The enriched population contained between 10-30% CD11C− pDC and 15 to 25% CD11c⁺ myeloid DC, identified on the expression of HLA-DR (tricolor, Becton Dickinson), CD11c (PE, Becton Dickinson) and lack of lineage markers (FITC) CD1a (Ortho Diagnostic System, Raritan, N.J.); CD14, CD15, CD57, CD16, CD20, CD3 (Becton Dickinson). For some experiments cells were further purified by Facs-sorting based on the above triple staining, and reanalysis of the sorted HLA-DR+, CD11c− and HLA-DR+, CD11c+ populations showed a purity higher than 95%.

Generation of DC from Cord Blood CD34⁺ HPC and Monocytes.

CD34⁺ cells isolated from cord blood mononuclear fractions through positive selection as described (Caux et al., 1990, Blood 75:2292-2298; Caux et al., 1996, J. Exp. Med. 184:695-706), were cultured in the presence of SCF, GM-CSF and TNFα and 5% AB⁺ human serum as described in Caux et al., 1996, J. Exp. Med. 184:695-706, in endotoxin-free complete medium consisting of RPMI 1640 (Gibco, Grand Island, N.Y.) supplemented with 10% (v/v) heat-inactivated fetal bovine serum (FBS) (Flow Laboratories, Irvine, UK), 10 mM Hepes, 2 mM L-glutamine, 100 μg/ml gentamicin (Schering-Plough, Levallois, France). Optimal conditions were maintained until day 6 by splitting these cultures at day 4 in the same conditions. Cells were routinely used at day 6 for migration experiments, chemokine receptor expression analysis and/or FACS sorting.

Monocytes purified by immunomagnetic depletion (Dynabeads, Dynal Oslo, Norway) as described in Dieu et al., 1998, J. Exp. Med. 188:1-14. Monocyte-derived dendritic cells were produced by culturing purified monocytes for 6-7 days in the presence of GM-CSF and IL-4 (Sallusto et al., 1994, J. Exp. Med. 179:1109-1118).

Enrichment for Mouse Plasmacytoid DC from Bone Marrow.

Mouse plasmacytoid DC were isolated from bone marrow, enriched by magnetic beads depletion and identified based on the triple staining, CD11b−, CD11c+, GR1+. Mouse pDC were used for migration assay in transwell experiments.

Chemotaxis Assays in Transwells

Migration assays were carried out using Transwell (6.5 mm diameter, COSTAR, Cambridge, Mass.) with 5×10⁵ cells/well. Enriched blood DC populations were first pre-incubated for 2 hours at 37° C. and then placed for 2 hours in 3 μm pore size inserts and the migration was revealed by triple staining gated on CD11c⁻/HLA-DR⁺/lineage⁻ and CD11c⁺/HLA-DR⁺/lineage⁻. Day 6 CD34⁺ HPC-derived DC precursors were incubated for 1 hour in 5 μm pore size inserts and migrating cells were analyzed by double staining either for CD1a and CD14. Monocytes and monocyte-derived DC were incubated for 2 hour in 5 μm pore size inserts and migration was revealed by CD14 and/or CD1a staining.

In some experiments, checkerboard analysis where CXCR4 and CXCR3 ligands were opposed in upper and lower wells, were performed. In other protocols pre-incubation experiments where the cells were first incubated in presence of CXCR4 or CXCR3 ligands for 1 hour before performing the migration assay to both receptor ligands were performed.

Culture of pDC with Inactivated Influenza Virus.

Cells (1×10⁶/ml) were pre-incubated in presence of paraformaldehyde inactivated Influenza virus (Beijing strain 262/95, 1 hemaglutination unit/ml) in complete medium, with or without IL-3, for 2 hours at 37° C. Cells were then wash 2 times in complete medium before migration assay in transwell.

Quantitative Real Time PCR (TaqMan) Analyses of Chemokine Receptor mRNA Expression.

Cells were prepared as described above, and total RNA was extracted by the guanidinium thiocyanate method as mentioned by the manufacturer (RNAgents total RNA isolation system, Promega). 4 μg of RNA were treated with DNase I (Boehringer, Mannheim,Germany) and reverse transcribed with oligo dT14-18 (Gibco BRL, Gaithersburg, Md.) and random hexamer primers (Promega, Madison, Wis.) using standard protocols. cDNA was diluted to a final concentration of 5 ng/μl. 10 μl of cDNA were amplified in the presence of 12.5 μl of TaqMan universal master mix (Perkin Elmer, Foster City, Calif.), 0.625 μl of gene-specific TaqMan probe, 0.5 μl of gene-specific forward and reverse primers, and 0.5 μl of water. As an internal positive control, 0.125 μl of 18S RNA-specific TaqMan probe and 0.125 μl of 18S RNA-specific forward and reverse primers were added to each reaction. Specific primers and probes for chemokines and chemokine receptors measured were obtained from Perkin Elmer. Gene-specific probes used FAM as reporter whereas probes for the internal positive control (18S RNA) were associated with either the JOE or VIC reporters. Samples underwent the following stages: stage 1, 50° C. for 2 minutes, stage 2, 95° C. for 10 minutes and stage 3, 95° C. for 15 seconds followed by 60° C. for 1 minute. Stage 3 was repeated 40 times. Gene-specific PCR products were measured by means of an ABI PRISM [symbol] 7700 Sequence Detection System (Perkin Elmer), continuously during 40 cycles. Specificity of primer probe combination was confirmed in cross-reactivity studies performed against plasmids of all known chemokine receptors (CCR1-CCR10, CXCR1-CXCR5, XCR1, CX3CR1). Target gene expression was normalized between different samples based on the values of the expression of the internal positive control.

Immunohistochemistry.

Frozen 6 μm tissue sections (human tonsils and skin) were fixed in acetone (and in 4% paraformaldehyde for MIP3α staining) before the immunostaining. To block the non-specific activities, sections were pre-treated with avidin D and biotin solutions (Blocking kit, Vector, Biosys SA, Compiègne, France) for 10 min each step and with 0.3% hydrogen peroxide (Sigma, Chemical Co., St Louis, Mo.) for 15 min at room temperature. After a brief washing in PBS, the sections were incubated with blocking serum (2% normal rabbit serum, same species than secondary antibody) for at least 30 min before adding both primary antibodies. Sections were immunostained with two (simultaneously) of the following antibodies: polyclonal anti-hMIP-3α (Goat IgG, R&D System Inc), anti-hMig (mlgG1,clone 49106.11, R&D System Inc), anti-hSDF1 (mlgG2a,clone K15C,Amara Ali, J. Biol. chem. 1999,vol274,p23916-23925) and anti-hMIP-3α (IgG1 206D9, R&D System Inc.), anti-hCD11c (IgG1, clone KB90, Dako, Glostrup, Denmark), anti-hE-cadherin (IgG1, HECD-1, Takara), anti-hCD105 (IgG1, clone266,Pharmingen) mouse monoclonal antibodies for 1 hour at room temperature in a humid atmosphere. The binding of goat IgG was detected by biotinylated rabbit anti-goat IgG followed by streptavidin-peroxydase both included in the Vectastain ABC kit (Goat IgG PK-4005, Vector), the binding of mouse IgG1 was revealed by rabbit alkaline phosphatase-labeled anti-mouse Ig (D0314, Dako) for 30 min at room temperature in a humid atmosphere. The peroxydase and alkaline phosphatase activities were revealed using 3-amino-9-ethylcarbazole (AEC) substrate (SK-4200, Vector) and alkaline phosphatase substrate III (SK-5300, Vector) for 1 to 10 min at room temperature, respectively. Negative controls were established by adding nonspecific isotype controls as primary antibodies.

### Example 1

### Despite Expression of Receptors for Inflammatory Chemokines, Plasmacytoid DC respond to the constitutive chemokine SDF-1

pDC were enriched from PBMC by magnetic beads depletion. Chemokine receptor and other marker expression was determined by triple staining on enriched blood DC populations and gating on Lin−, CD11c− (FITC), HLA-DR+ (tricolor), using PE-coupled antibodies. Following this protocol, the CD11c− pDC were 95-98% CD45RA+ and IL-3Rα+. pDC expressed CCR2 and CCR5 (FIG. 1) at a comparable levels to CD11c+ circulating blood DC (Vanbervliet et al., 2001, Eur J Immunol. 32(1):231-42). CCR1, CCR3, CCR4, CCR6, CCR7, CXCR1, CXCR2, CXCR5 were not significantly expressed as detected by cytofluorimetry (FIG. 1) and/or RT-PCR.

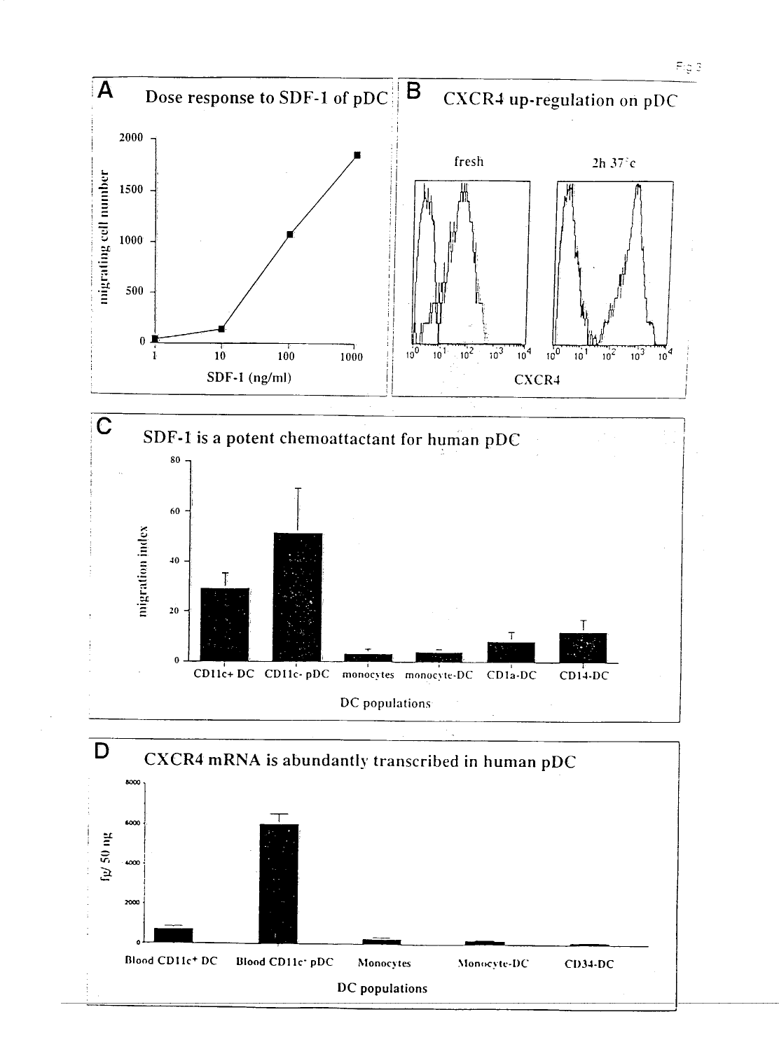

To determine migration of pDC in response to various chemokines, circulating blood DC subsets were enriched by magnetic bead depletion. After purification, cells were rested for 2 hours at 37° C. and studied in transwell (5 μm pore size) migration assay. The migration was revealed after 2 hours by triple staining: lineage markers FITC, HLA-DR tricolor, and CD11c PE, and analyzed by Facs. As shown in FIG. 2, pDC only marginally responded to CCR2 (MCPs) and CCR5 (RANTES) ligands compared to blood CD11c+ DC. In contrast, as shown in FIGS. 2 and 3, pDC migrated very efficiently in response to SDF-1, with an IC50 observed around 100 ng/ml SDF-1 (FIG. 3A).

Next, various DC populations were analyzed for their response to SDF-1 over a wide range of concentrations (1 to 1000 ng/ml). Circulating blood CD11c− pDC and myeloid CD11c+ DC were enriched by magnetic bead depletion, and studied in a transwell (3 μm pore size) migration assay as described above. Monocytes and monocyte-derived DC (7 days in presence of GM-CSF+IL-4) were tested in transwell (5 μm pore size) migration assay, revealed after 2 hours by CD14/CD1a double staining. CD34⁺ HPC were cultured in presence of SCF, GM-CSF, TNF-α and 5% human serum for 6-7 days and used in transwell (5μ pore size) migration assays (5×10⁵ cells/well). After 1 hour, migration was revealed by double color staining for CD1a and CD14, and analyzed by Facs. Compared to other DC subsets, SDF-1 was highly and more active on pDC as compared to other DC populations(FIG. 3C).

CXCR4 mRNA expression was next analyzed by quantitative RT-PCR. Cells were prepared as described above, except for blood CD11c− pDC and myeloid CD11c⁺, which were isolated by Facs-sorting based on CD11c, HLA-DR expression and lack of lineage markers. Cells were recovered, RNA extracted, DNAse treated, reverse transcribed and quantitative PCR for CXCR4 was performed. High levels of CXCR4 mRNA were detected, as shown in FIG. 3D. In addition, expression of CXCR4 was rapidly (2 hours) up-regulated at cell surface of pDC (FIG. 3B).

SDF-1 was very potent in inducing freshly isolated pDC migration. This potent activity of SDF-1 was in line with very high levels of CXCR4 mRNA expression compared to other DC populations. In addition, CXCR4 protein already detected at the cell surface after isolation was very rapidly translocated at the cell surface at 37° C. It is likely that CXCR4 protein is stored in intracytoplasmic compartments in these cells, as previously described in other cell types (Forster et al., 1998, J. Immunol. 160(3):1522-31; Cole et al., 1999, J. Immunol. 162(3):1392-400).

### Example 2

### Plasmacytoid DC Express High Levels of CXCR3 Compared to other DC Populations

For blood CD11c− pDC, chemokine receptor and other marker expression was determined by triple staining on enriched blood DC populations and gating on Lin−, CD11c− (FITC), HLA-DR+ (tricolor), using PE-coupled antibodies. Following this protocol the CD11c− pDC were 95-98% CD45RA+ and IL-3Rα+.

For blood CD11c+ myeloid DC, chemokine receptor and other marker was determined by triple staining gated on Lin−, CD45RA− (FITC), HLA-DR+ (tricolor), using PE-coupled antibodies. Following this protocol the CD11c+ myeloid DC were 95-98% CD11c+, IL-3Rα−.

CD34-derived DC or Monocyte-derived DC were processed for double staining using FITC-conjugated CD1a or CD14 and PE-conjugated monoclonal antibodies against human chemokine receptors.

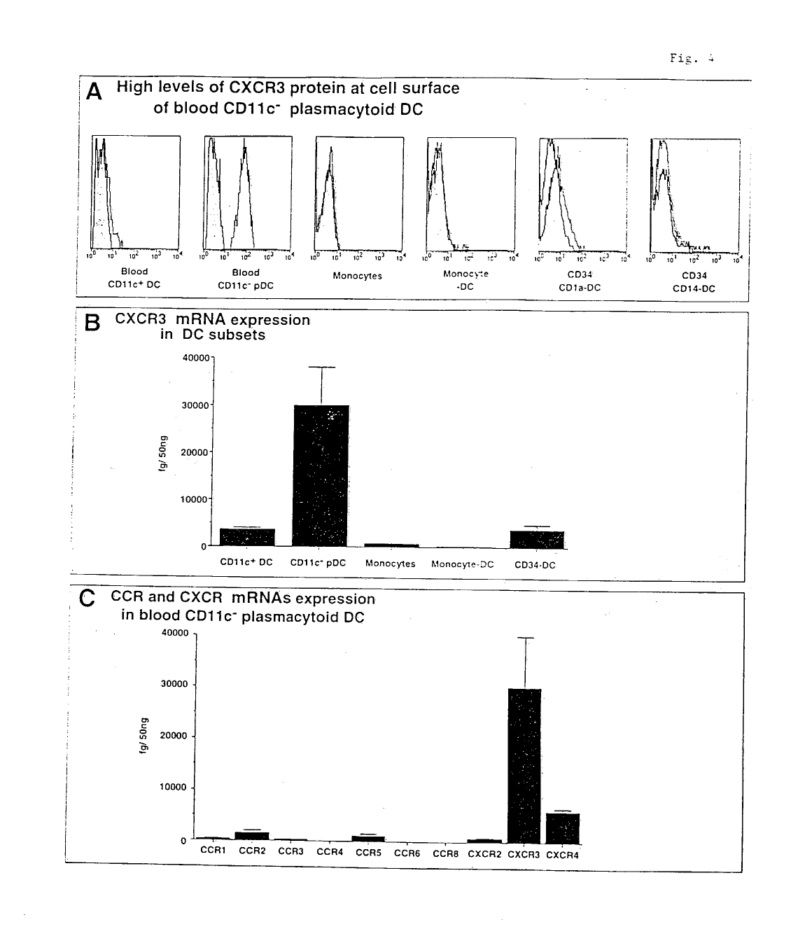

As shown in FIG. 1, pDC expressed high levels of CXCR3 at cell surface. In contrast, circulating CD11c+ blood DC, as well as other DC populations, did not express significant levels of CXCR3, as detected by FACS or by quantitative RT-PCR according to the method disclosed in Example 1 (FIG. 4A&B).

mRNA expression of CXCR3 was next analyzed as described in Example 1. Compared to other chemokine receptors, CXCR3 mRNA was the receptor expressed at the highest level on pDC (FIG. 4C), even higher than CXCR4 mRNA.

Given the results described above regarding the high level of expression of CXCR3 receptors on pDC, the CXCR3 ligands IP-10, Mig and I-TAC were next tested in the chemotaxis assays described above. Contrary to what was expected, only a marginal migration was observed (FIG. 2), and only at high concentration (FIG. 5, 1-5 μg/ml), even after contact with viruses (see Example 9), or in trans-endothelial migration assays.

### Example 3

### CXCR3 Ligands Synergize with SDF-I to Induce Potent Migration of pDC

Migration assays were performed in response to different SDF-1 and CXCR3 ligand combinations.

As shows in FIG. 5, in presence of sub-optimal dose of SDF-1 (10 ng/ml) the activity of all 3 CXCR3-ligands was observed at lower concentration (100-500 ng/ml) (FIG. 5B). In addition, when tested in combination with SDF-1, all 3 CXCR3-ligands allowed to lower the threshold of SDF-1 sensitivity by 2 order of magnitude.

### Example 4

### CXCR3 Ligands Prime Human CD11c− pDC by Increasing Their Sensitivity to SDF-1

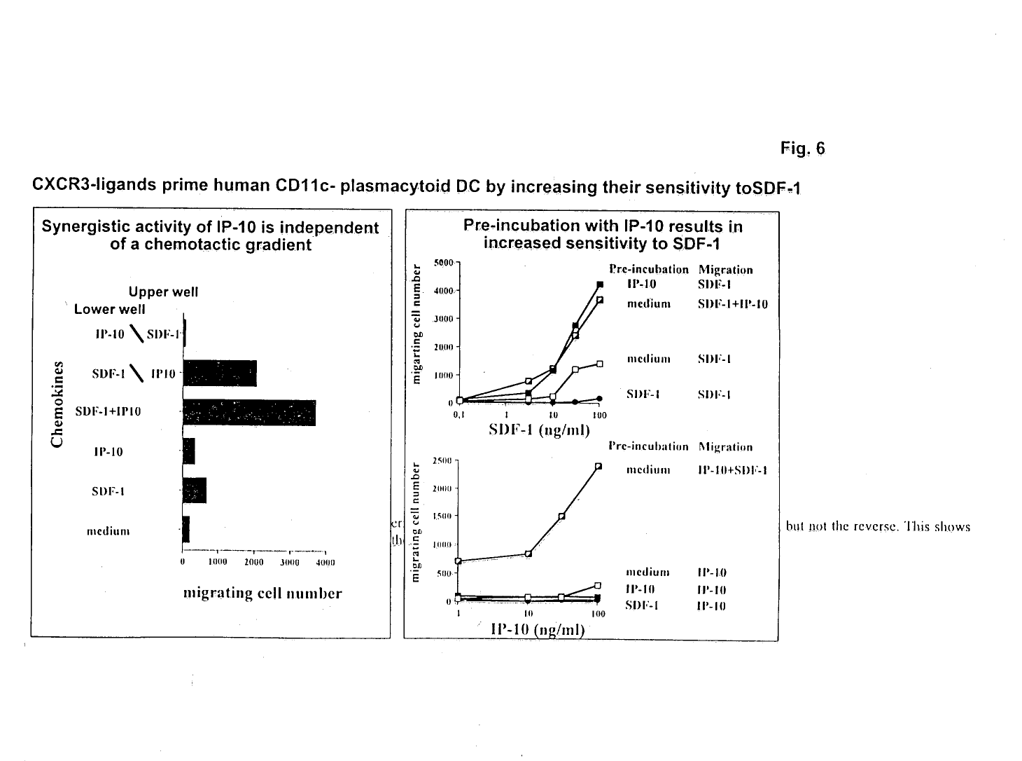

Checkerboard analysis where CXCR4 and CXCR3 ligands were opposed in upper and lower wells, were performed. Synergystic activity was observed when the two chemokines were placed together in the lower well, as well as when IP-10 was in the upper well together with pDC, and SDF-1 in the lower well, but not the reverse (FIG. 6A). Then pre-incubation experiments, where the cells were first incubated in presence of CXCR4 or CXCR3 ligands for 1 hour before performing the migration assay to both receptor ligands were performed. When the cells were first primed with IP-10, an increased response to SDF-1 was observed, but not in the reverse experiment (FIG. 6B).

These results suggest that CXCR3-L activity is independent of the gradient and that they sensitize pDC to respond to lower SDF-1 concentrations. Finally, these observations also demonstrate that the synergistic activity results from a sequential action, with CXCR3 ligands acting first and SDF-1 acting second. These conclusions are in agreement with the observed expression of CXCR3 ligands and SDF-1 expression in vivo at site of inflammation (see Example 8).

### Example 5

### CXCR3 Ligands and SDF-1 Induce Mouse pDC Migration

Mouse plasmacytoid DC were isolated from bone marrow, enriched by magnetic beads depletion and identified based on the triple staining, CD11b−, CD11c+, GR1+. Mouse pDC were used for migration assay in transwell experiments.

When tested on the recently identified mouse pDC, CXCR3 ligands IP-10, MIG and I-TAC alone induced their migration in transwell assays (FIG. 7). The level of migration induced with CXCR3-ligands was comparable to that observed with SDF-1, but the selectivity of CXCR3-ligands was much more important than that of SDF-1.

### Example 6

### Plasmacytoid DC Express High Levels of L-Selectin Compared to to Other DC Populations, but They Also Express CLA

pDC have been shown to express CD62L (Celia et al., 1999, Nature Med. 5:919-923). Here we compared the expression of L-selectin on different DC populations. For blood CD11c− pDC, the analysis of L-selectin and CLA expression was performed on the enriched DC population by triple staining: lin⁻ CD11c⁻ (FITC), HLA-DR⁺ (Tricolor) and anti-CD62L or CLA (PE). For blood CD11c⁺ myeloid DC the expression was determined by triple staining: In⁻ CD45RA⁻ (FITC), HLA-DR⁺ (Tricolor) and anti-CD62L or CLA (PE). For monocytes, and monocyte-derived DC, the analysis was obtained by double staining against anti-CD14 antibody or anti-CD1a (FITC), respectively. For CD34⁺ HPC-derived CD1a⁺ and CD14⁺ DC precursors, double staining with anti-CD62L or CLA (PE) and CD1a or CD14 antibodies (FITC).

As can be seen in FIG. 8, we found that upon isolation, pDC expressed very high levels of L-selectin, at a density comparable to that of naive T cells. In contrast, CD11c+ blood DC expressed 20 to 50 fold lower levels of L-selectin comparable to that of circulating monocytes. In vitro generated DC from monocytes or CD34+ precursors did not express significant levels of L-selectin. In addition, after 2 to 16 hours culture CD62-L expression was maintained on pDC while it disappeared on CD11c+ DC.

These observations suggest that pDC may have the capacity to enter lymph nodes from the blood through the HEV like naive T cells. However, pDC also expressed the cutaneous homing molecule CLA, at a density similar to that expressed on most other circulating DC and monocytes (FIG. 4B), suggesting that they might also have the capacity to enter non-lymphoid tissue.

### Example 7

### CCR6 and CCR10 Expression on Human pDC and Migration to Their Respective Ligands is Induced upon Culture in IL-3

Plasmacytoid DC isolated by Facs-sorting, were cultured in presence of IL-3 and other survival factors (PFA inactivated influenza virus, ODN, CD40L) or combinations for 24 to 72 hours.

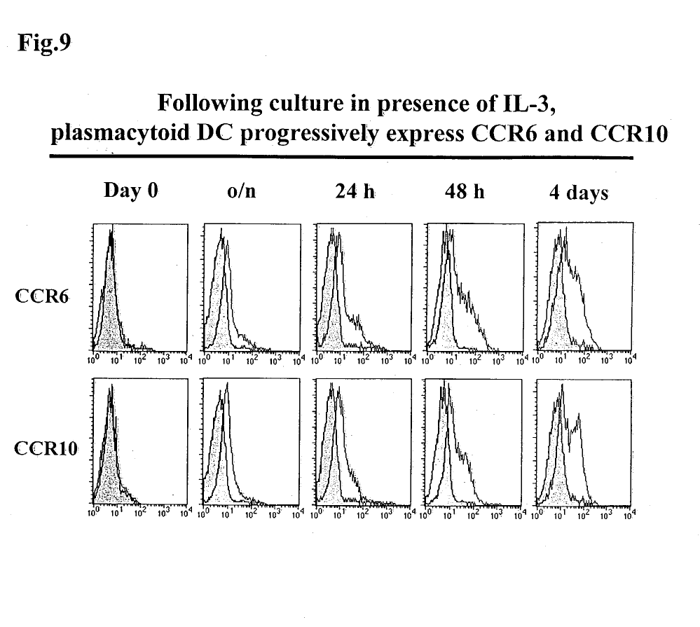

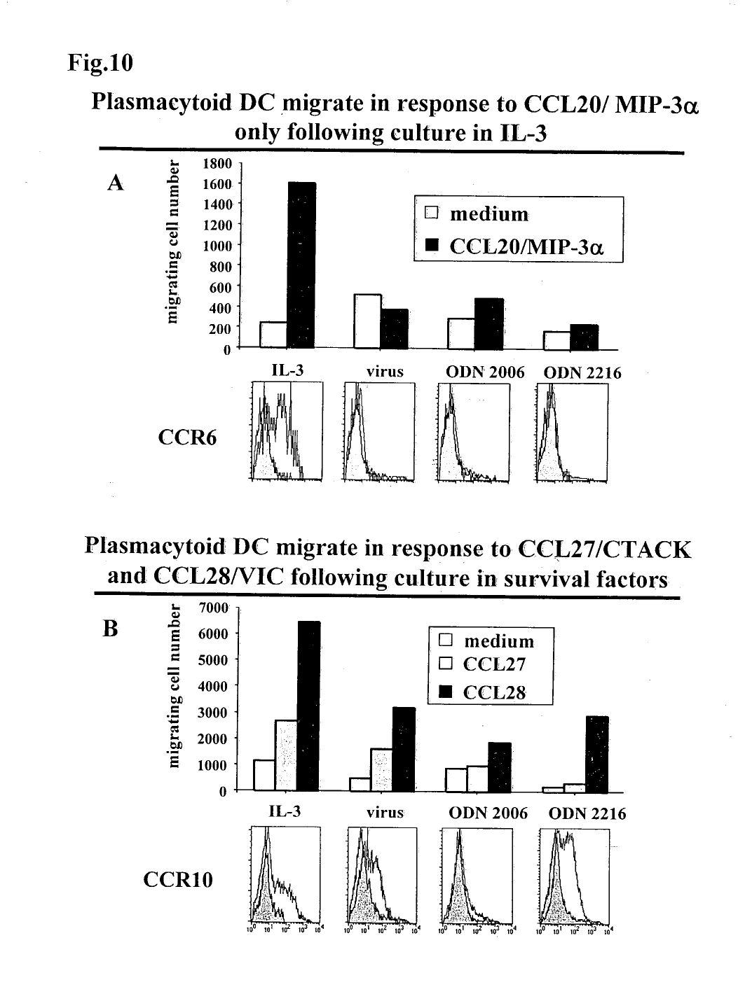

When cultured in the presence of IL-3 (FIG. 9) or IL-3+CD40-L, human pDC specifically acquired the expression of CCR6 and CCR10, but not that of other receptors and lost the expression of receptors present upon isolation. Upon culture in IL-3, pDC strongly migrate in transwell migration assays in response to CCR6 and CCR10 ligands, CCL20 and CCL27/CCL28, respectively (FIG. 10A,B). pDC cultured in IL-3 start to respond to CCL20 from 10 ng/ml while higher CCR10-ligands were required (1 μg/ml), as previously reported for memory T cells (Morales et al., 1999, PNAS 96(25):14470-5; Hudak et al., 2002, J Immunol. 169(3):1189-96). The expression of CCR6 and the response to CCL20 was only induced by IL-3 (FIG. 10A), while CCR10 expression and response to its ligands was induced by other survival factors such as virus and ODN (FIG. 10B). This might suggest that CCR10 expression might be part of a differentiation program during pDC life cycle, and might have an important physiological role in the control of pDC trafficking. The expression of CCR6 appears more tightly regulated and might play a role in the fine positioning of pDC in tissues.

### Example 8

### Mig Expressed by Endothelial Cells Form Complementary Gradients with SDF-1, CTACK and MIP-3α

Immunohistochemistry on tonsil and inflammed skin (psoriatic lesions) was performed using antibodies against the different chemokines.

In inflamed skin, Mig was expressed in vessels in dermal papilla, in the vinicity of epithelial cells expressing CTACK and MIP-3α. Similarly, in tonsil, Mig was expressed by blood vessels in contact with epithelial cells were SDF-1 and MIP-3α form complementary gradients.

### Example 9

### Upon Contact with Virus, pDC Acquire CCR7 Expression and CCR7 Ligands Activity and Rapidly Lose L-Selectin Expression

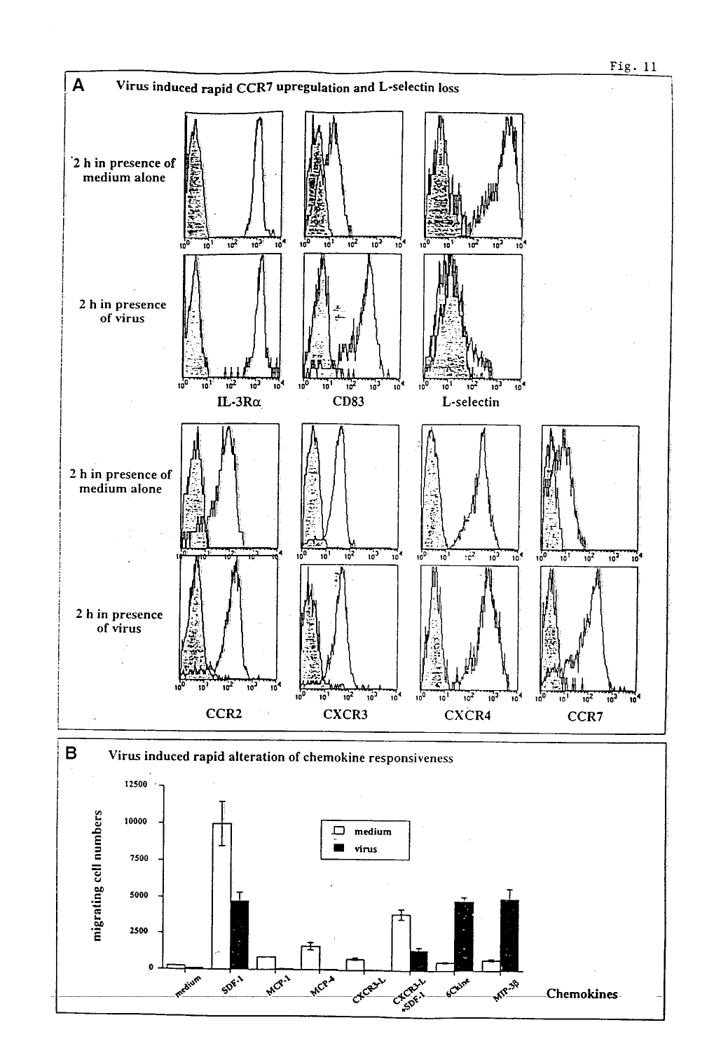

As pDC are known to be key mediators of IFNα production upon encounter with viruses (Siegal et al., 1999, Science 284(5421):1835-7; Cella et al., 1999, Nature Med. 5:919-923), chemokine receptor expression and chemokine responsiveness of pDC was next assessed after exposure, for 2 to 16 hours, to PFA inactivated influenza virus. After 2 hours contact with virus, the levels of CCR2, CCR5, CXCR3 and CXCR4 expression remained unchanged (FIG. 11A), or slightly increased, but the response to CCR2 and CXCR3-ligands was totally abolished (FIG. 11B), while SDF-1 was still active (less than 50% loss of activity). After 16 hours, both CCR2, CCR5, CXCR3, CXCR4 receptor expression and ligand responsiveness were lost. In contrast, already after 2 hours in presence of virus, CD83 and CCR7 up-regulation were clearly observed (FIG. 11A), and were accentuated after 16 hours. In parallel to CCR7 induced expression, 6Ckine and MIP-3β induced a potent migration of virus activated pDC at 2 (FIG. 11B) and 16 hours, while no or marginal migration of non-activated pDC was observed. For both ligands the optimal active concentration was 100 ng/ml.

This observation suggests that following local recruitment and activation these cells will have the capacity to emigrate in the lymph node through the lymphatic stream, a process controlled by CCR7 and its ligands.

Thus, combinations of chemokine allowing pDC recruitment, together with signals inducing pDC activation, will empower pDC to emigrate in the lymph node and to initiate immune response in particular Th-1 type immune responses through IFNα production.

Taken together, these results suggest that in addition to the ability to percolate to the lymph node from blood through high endothelial venule, pDC may have the capacity to reach inflamed tissues through CLA expression. This recruitment in non-lymphoid tissues likely requires the sequential action of different chemokine gradients. First, CXCR3 ligands in concert with CXCR4 ligands induce recruitment of pDC from blood to tissue. Then, signals from the microenvironment (for example, IL-3 from mast cells) may induce CCR6 and/or CCR10 expression, allowing pDC to reach the site of virus entry, the epithelium, where CCR6 and CCR10 ligands are produced. Alternatively, as a soluble mediator, IL-3 may reach the blood allowing CCR6/10 expression on circulating pDC and their direct recruitment from blood to tissues through CCR6/10 ligands.

In summary, the results reported herein support the use of the chemokine receptor agonists set forth above, alone or in combination with each other, a survival factor and/or a disease associated antigen, with or without an activating agent to recruit pDC either locally at the site of chemokine injection, or directly into tumors. Also supported by these results is the use of the chemokine receptor antagonists set forth above, alone or in combination with each other to block the migration of pDC.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method of treating a disease state comprising administering to an individual in need thereof an amount of a chemokine receptor agonist or antagonist sufficient to increase or decrease the migration of plasmacytoid dendritic cells.

2. A method of treating a disease state comprising administering to an individual in need thereof an amount of a chemokine receptor agonist sufficient to enhance or modulate an immune response, wherein the chemokine receptor agonist is selected from the group consisting of a CXCR3 agonist, a CXCR4 agonist, a CCR6 agonist, and a CCR10 agonist, or a combination thereof.

3. The method of claim 2 wherein the chemokine receptor agonist is selected from the group consisting of IIP-10, Mig, I-TAC, SDF-1, MIP-3α, MEC and CTACK.

4. The method of claim 2 wherein the chemokine receptor agonist is recombinant.

5. The method of claim 2 wherein the chemokine receptor agonist is a small molecule.

6 The method of claim 2 wherein the chemokine receptor agonist is administered in combination with one or more other chemokine receptor agonists.

7. The method of claim 2 wherein the disease state is a bacterial infection, a viral infection, a fungal infection, a parasitic infection or cancer.

8. The method of claim 2 wherein the disease state is an autoimmune disorder, allergy, or transplantation.

9. The method of claim 7 further comprising administering at least one disease-associated antigen.

10. The method of claim 9 wherein the antigen is a tumor-associated antigen.

11. The method of claim 9 wherein the antigen is a bacterial, viral or fungal antigen.

12. The method of claim 9 wherein the antigen is a self antigen, an histocompatability antigen or an allergen.

13. The method of claim 10 wherein the tumor-associated antigen is selected from the group consisting of Melan-A, tyrosinase, p97, β-HCG, GalNAc, MAGE-1, MAGE-2, MAGE-3, MAGE-4, MAGE-12, MART-1, MUC1, MUC2, MUC3, MUC4, MUC18, CEA, DDC, melanoma antigen gp75, HKer 8, high molecular weight melanoma antigen, K19, Tyr1 and Tyr2, members of the pMel 17 gene family, c-Met, PSA, PSM, α-fetoprotein, thyroperoxidase, and gp100.

14. The method of claim 10 wherein the disease state to be treated is cancer selected from the group consisting of cancers affecting the oral cavity, pharynx, disgestive system, respiratory system, head and neck, bones and joints, soft tissues, skin, breast, genital system, urinary system, eye and orbit, brain and nervous system, endocrine system, and blood/hematopoietic system.

15. The method of claim 10 wherein the cancer to be treated is prostate cancer and the tumor-associated antigen is PSA and/or PSM.

16. The method of claim 10 wherein the disease state to be treated is melanoma and the tumor-associated antigen is Melan-A, gp100 or tyrosinase.

17. The method of claim 2 further comprising administering a survival factor.

18. The method of claim 17, wherein the survival factor is selected from the group consisting of IL-3, IFNα and RANK-L.

19. The method of claim 7 further comprising administering an activating agent.

20. The method of claim 19 wherein the activating agent is selected from the group consisting of IFNα, TNFα, RANK ligand/agonist, CD40 ligand/agonist and a Toll-like receptor ligand/agonist.

21. The method of claim 2 wherein the chemokine receptor agonist(s) are administered intradermally, intramuscularly, subcutaneously, intra-venously, per os, topically, or in the form of a vector.

22. The method of claim 2 wherein the chemokine receptor agonist is administered in the form of a targeting construct comprising a chemokine receptor agonist and a targeting moiety, wherein the targeting moiety is a peptide, a protein, an antibody, an antibody fragment, a small molecule, or a vector which is engineered to recognize or target a disease-associated antigen.

23. The method of claim 2 wherein the chemokine receptor agonist is administered in the form of a fusion protein.

24. The method of claim 23 whereing the fusion protein also comprises a disease associated antigen.

25. A method of treating a disease state comprising administering to an individual in need thereof an effective amount of a CXCR3 agonist in combination with an effective amount of a CXCR4 agonist.

26. The method of claim 25 wherein the CXCR4 agonist is SDF-1 or a biologically active fragment thereof and the CXCR3 agonist is selected from the group consisting of IP-10, MIG, I-TAC, and biologically active fragments thereof.

27. The method of claim 25 wherein the CXCR3 agonist or the CXCR4 agonist is recombinant.

28. The method of claim 25 wherein the CXCR3 agonist or the CXCR4 agonist is a small molecule.

29. The method of claim 25 wherein the disease state is a bacterial infection, a viral infection, a fungal infection, a parasitic infection or cancer.

30. The method of claim 29 further comprising administering at least one dsease-associated antigen.

31. The method of claim 30 wherein the antigen is a tumor-associated antigen.

32. The method of claim 30 wherein the antigen is a bacterial, viral or fungal antigen.

33. The method of claim 30 wherein the antigen is a self antigen, a histocompatibility antigen or an allergen.

34. The method of claim 31 wherein the tumor-associated antigen is selected from the group consisting of Melan-A, tyrosinase, p97, β-HCG, GalNAc, MAGE-1, MAGE-2, MAGE-3, MAGE-4, MAGE-12, MART-1, MUC1, MUC2, MUC3, MUC4, MUC18, CEA, DDC, melanoma antigen gp75, HKer 8, high molecular weight melanoma antigen, K19, Tyr1 and Tyr2, members of the pMel 17 gene family, c-Met, PSA, PSM, α fetoprotein, thyroperoxidase, and gp100.

35. The method of claim 25 wherein the disease state to be treated is cancer selected from the group consisting of cancers affecting the oral cavity and pharynx, disgestive system, respiratory system, head and neck, bones and joints, soft tissues, skin, breast, genital system, urinary system, eye and orbit, brain and nervous system, endocrine system, and blood/hematopoietic system.

36. The method of claim 35 wherein the cancer to be treated is prostate cancer and the tumor-associated antigen is PSA and/or PSM.

37. The method of claim 35 wherein the disease state to be treated is melanoma and the tumor-associated antigen is Melan-A, gp100 or tyrosinase.

38. The method of claim 25 further comprising administering a survival factor.

39. The method of claim 38, wherein the survival factor is selected from the group consisting of IL-3, IFNα and RANK-L.

40. The method of claim 25 further comprising administering an activating agent.

41. The method of claim 40 wherein the activating agent is selected from the group consisting of IFNα, TNFα, RANK ligand/agonist, CD40 ligand/agonist, and a Toll-like receptor ligand/agonist.

42. The method of claim 25 wherein the chemokine receptor agonist(s) are administered intradermally, intramuscularly, subcutaneously, intra-venously, per os, topically, or in the form of a vector.

43. The method of claim 25 wherein the chemokine receptor agonist(s) are administered in the form of a targeting construct comprising a chemokine receptor agonist and a targeting moiety, wherein the targeting moiety is a peptide, a protein, an antibody, an antibody fragment, a small molecule, or a vector which is engineered to recognize or target a disease-associated antigen.

44. The method of claim 25 wherein the chemokine receptor agonist(s) are administered in the form of a fusion protein.

45. The method of claim 44, wherein the fusion protein further comprises a tumor associated antigen.

46. A method of treating a disease state comprising administering to an individual in need thereof an effective amount of a CCR6 agonist and/or a CCR10 agonist in combination with an effective amount of a survival factor.

47. The method of claim 46 wherein the CCR6 agonist is MIP-3α or a variant thereof and the CCR10 agonist is CTACK, MEP or a variant thereof.

48. The method of claim 46 wherein the CCR6 agonist or the CCR10 agonist is recombinant.

49. The method of claim 46 wherein the CCR6 agonist or the CCR10 agonist is a small molecule.

50. The method of claim 46 wherein the survival factor is selected from the group consisting of IL-3, IFNα and RANK-L.

51. The method of claim 46 further comprising administering an activating agent.

52. The method of claim 51 wherein the activating agent is selected from the group consisting of IFNα, TNFα, RANK ligand/agonist, CD40 ligand/agonist or a Toll-like receptor ligand/agonist.

53. The method of claim 46 wherein the disease state is a bacterial infection, a viral infection, a fungal infection, a parasitic infection or cancer.

54. The method of claim 46 further comprising administering at least one disease-associated antigen.

55. The method of claim 54 wherein the antigen is a tumor-associated antigen.

56. The method of claim 54 wherein the antigen is a bacterial, viral or fungal antigen.

57. The method of claim 54 wherein the antigen is a self antigen, a histocompatibility antigen or an allergen.

58. The method of claim 55 wherein the tumor-associated antigen is selected from the group consisting of Melan-A, tyrosinase, p97, P-HCG, GalNAc, MAGE-1, MAGE-2, MAGE-3, MAGE-4, MAGE-12, MART-1, MUC1, MUC2, MUC3, MUC4, MUC18, CEA, DDC, melanoma antigen gp75, HKer 8, high molecular weight melanoma antigen, K19, Tyr1 and Tyr2, members of the pMel 17 gene family, c-Met, PSA, PSM, α fetoprotein, thyroperoxidase, and gp100.

59. The method of claim 53 wherein the disease state to be treated is cancer selected from the group consisting of cancers affecting the oral cavity and pharynx, disgestive system, respiratory system, head and neck, bones and joints, soft tissues, skin, breast, genital system, urinary system, eye and orbit, brain and nervous system, endocrine system, and blood/hematopoietic system.

60. The method of claim 59 wherein the cancer to be treated is prostate cancer and the tumor-associated antigen is PSA and/or PSM.

61. The method of claim 59 wherein the disease state to be treated is melanoma and the tumor-associated antigen is Melan-A, gp100 or tyrosinase.

62. The method of claim 46 wherein the chemokine receptor agonist(s) are administered intradermally, intramuscularly, subcutaneously, intra-venously, per os, topically, or in the form of a vector.

63. The method of claim 46 wherein the chemokine receptor agonist is administered in the form of a targeting construct comprising a chemokine receptor agonist and a targeting moiety, wherein the targeting moiety is a peptide, a protein, an antibody, an antibody fragment, a small molecule, or a vector which is engineered to recognize or target a disease-associated antigen.

64. The method of claim 46 wherein the chemokine receptor agonist is administered in the form of a fusion protein.

65. The method of claim 64 wherein the fusion protein further comprises a disease associated antigen.

66. A method of treating a disease state comprising administering to an individual in need thereof an effective amount of a CCR6 agonist and/or a CCR10 in combination with an effective amount of a CXCR3 agonist and a survival factor.

67. The method of claim 66 wherein the CCR6 agonist is MIP-3α, or a biologically active fragment thereof, the CCR10 agonist is CTACK or a biologically active fragment thereof, and the CXCR3 agonist is selected from the group consisting of IP-10, Mig, I-TAC, and biologically active fragments thereof.

68. The method of claim 66 wherein CCR6 agonist or the CCR10 agonist or the CXCR3 agonist is recombinant.

69. The method of claim 66 wherein the CCR6 agonist or the CCR10 agonist or the CXCR3 agonist is a small molecule.

70. The method of claim 66, wherein the survival factor is selected from the group consisting of IL-3, IFNα and RANK-L.

71. The method of claim 66 further comprising administering an activating agent.

72. The method of claim 71 wherein the activating agent is selected from the group consisting of IFNα, TNFα, RANK ligand/agonist, CD40 ligand/agonist and a Toll-like receptor ligand/agonist.

73. The method of claim 66 wherein the disease state is a bacterial infection, a viral infection, a fungal infection, a parasitic infection or cancer.

74. The method of claim 66 further comprising administering at least one disease-associated antigen.

75. The method of claim 74 wherein the antigen is a tumor-associated antigen.

76. The method of claim 74 wherein the antigen is a bacterial, viral or fungal antigen.

77. The method of claim 74 wherein the antigen is a self antigen, a histocompatability antigen or an allergen.

78. The method of claim 75 wherein the tumor-associated antigen is selected from the group consisting of Melan-A, tyrosinase, p97, β-HCG, GalNAc, MAGE-1, MAGE-2, MAGE-3, MAGE-4, MAGE-12, MART-1, MUC1, MUC2, MUC3, MUC4, MUC18, CEA, DDC, melanoma antigen gp75, HKer 8, high molecular weight melanoma antigen, K19, Tyr1 and Tyr2, members of the pMel 17 gene family, c-Met, PSA, PSM, α-fetoprotein, thyroperoxidase, and gp100.

79. The method of claim 73 wherein the disease state to be treated is cancer selected from the group consisting of cancers affecting the oral cavity and pharynx, disgestive system, respiratory system, head and neck, bones and joints, soft tissues, skin, breast, genital system, urinary system, eye and orbit, brain and nervous system, endocrine system, and blood/hematopoietic system.

80. The method of claim 79 wherein the cancer to be treated is prostate cancer and the tumor-associated antigen is PSA and/or PSM.

81. The method of claim 79 wherein the disease state to be treated is melanoma and the tumor-associated antigen is Melan-A, gp100 or tyrosinase.

82. The method of claim 66 wherein the chemokine receptor agonist(s) are administered intradermally, intramuscularly, subcutaneously, intra-venously, per os, topically, or in the form of a vector.

83. The method of claim 66 wherein the chemokine receptor agonist is administered in the form of a targeting construct comprising a chemokine receptor agonist and a targeting moiety, wherein the targeting moiety is a peptide, a protein, an antibody, an antibody fragment, a small molecule, or a vector which is engineered to recognize or target a disease-associated antigen.

84. The method of claim 66 wherein the chemokine receptor agonist is administered in the form of a fusion protein.

85. The method of claim 84 wherein the fusion protein further comprises a disease associated antigen.

86. A method of treating a disease state comprising administering to an individual in need thereof an effective amount of a chemokine receptor antagonist sufficient to decrease migration of pDC, wherein the chemokine receptor antagonist is selected from the group consisting of a CXCR3 antagonist, a CXCR4 antagonist, a CCR6 antagonist, and a CCR10 antagonist, or a combination thereof.

87. The method of claim 86 wherein the disease state is an autoimmune disease, graft rejection or allergy.

88. The method of claim 86 wherin the disease state is cancer or an infectious disease.

89. The method of claim 86 wherein the chemokine receptor antagonist is recombinant.

90. The method of claim 86 wherein the chemokine receptor antagonist is a small molecule.

91. The method of claim 86 wherein the chemokine receptor antagonist(s) are administered intradermally, intramuscularly, subcutaneously, intravenously, per os, topically, or in the form of a vector.

92. The method of claim 86 wherein the chemokine receptor antagonist is administered in the form of a targeting construct comprising a chemokine receptor antagonist and a targeting moiety, wherein the targeting moiety is a peptide, a protein, an antibody, an antibody fragment, a small molecule, or a vector which is engineered to recognize or target a disease-associated antigen.
